(19) United States
(12) Patent Application Publication
(10) Pub. No.: US 2015/0094563 A1
(43) Pub. Date: Apr. 2, 2015

(54) MAGNETIC RESONANCE SYSTEM AND PROGRAM

(71) Applicant: GE MEDICAL SYSTEMS GLOBAL TECHNOLOGY COMPANY, LLC, Waukesha, WI (US)

(72) Inventors: Yuko Suwa, Tokyo (JP); Naoyuki Takei, Tokyo (JP)

(73) Assignee: GE MEDICAL SYSTEMS GLOBAL TECHNOLOGY COMPANY, LLC, Waukesha, WI (US)

(21) Appl. No.: 14/501,119

(22) Filed: Sep. 30, 2014

(30) Foreign Application Priority Data
Sep. 30, 2013 (JP) 2013204940

(51) Int. Cl. A61B 5/026; G01R 33/38; G01R 33/50; G01R 33/30; A61B 5/00; A61B 5/0205

(52) U.S. Cl. CPC A61B 5/0263; A61B 5/7285; A61B 5/0205; A61B 5/743; G01R 33/50; G01R 33/307; G01R 33/38; A61B 5/024
USPC 600/413; 600/419

(57) ABSTRACT

A magnetic resonance system configured to repeatedly execute imaging sequences each having a first RF pulse for flipping each spin in a region containing blood, and a data acquisition sequence acquiring data of the blood from the region is provided. The magnetic resonance system includes a storage unit configured to store a correspondence relation between a contrast between the blood and a background tissue, a first time taken from the first RF pulse to the data acquisition sequence, and a second time taken from a completion of an imaging sequence to a start of a next imaging sequence, first determining means configured to determine the first time used, and second determining means configured to determine the second time used.

### CROSS REFERENCE TO RELATED APPLICATIONS

This application claims the benefit of Japanese Patent Application No. 2013-204940 filed Sep. 30, 2013, which is hereby incorporated by reference in its entirety.

### BACKGROUND OF THE INVENTION

The present invention relates to a magnetic resonance system that acquires data of blood, and a program applicable to the magnetic resonance system.

As a method of imaging a blood flow, there has been known a method using an inflow effect (refer to Japanese Unexamined Patent Publication No. 2008-086748).

Further, as a method for acquiring a blood vessel image using an inflow effect, there has been known a method using an IFIR (Inhance Inflow IR) sequence. In the IFIR sequence, an inversion pulse is applied to invert magnetization of a region where imaging of blood vessel is desired. Then, data acquisition is executed in wait for the elapse of an inversion time since the application of the inversion pulse. In this method, since the blood having sufficiently large vertical magnetization flows into the region where imaging of blood vessel is desired during a period from the application of the inversion pulse to the acquisition of data, it is possible to obtain an image in which the blood vessel is drawn.

In this method, since the blood vessel is imaged using the blood inflow effect, the blood having sufficiently large vertical magnetization is required to flow into the entire region where imaging of the blood vessel is desired, during the period from the application of the inversion pulse to the acquisition of the data. However, depending on the set value of the inversion time, the blood having sufficiently large vertical magnetization cannot be sufficiently made flow into the entire region where imaging of the blood vessel is desired. Therefore, there can be a case where a desired blood vessel image cannot be obtained. Also, a problem arises in that when the inversion time is made too long, a background tissue is recovered before the data acquisition, so that the contrast of an image is deteriorated.

There has thus been a demand for an imaging method capable of making the blood with sufficiently large vertical magnetization flow into the entire region where imaging of the blood vessel is desired, and further capable of enhancing the contrast of the image sufficiently.

### BRIEF DESCRIPTION

In a first aspect, a magnetic resonance system which repeatedly executes imaging sequences each having a first RF pulse for flipping each spin in a region containing blood, and a data acquisition sequence acquiring data of the blood from the region is provided. The magnetic resonance system includes a storage unit that stores therein a correspondence relation between a contrast between the blood and a background tissue, a first time taken from the first RF pulse to the data acquisition sequence, and a second time taken from the completion of an imaging sequence to the start of the next imaging sequence, first determining means that determines the first time used when the imaging sequence is repeatedly executed, based on a flow velocity of the blood, and second determining means that determines the second time used when the imaging sequence is repeatedly executed, based on the first time determined by the first determining means and the correspondence relation.

In a second aspect, a program applied to a magnetic resonance system which repeatedly executes imaging sequences each having a first RF pulse for flipping each spin in a region containing blood, and a data acquisition sequence acquiring data of the blood from the region, and which stores therein a correspondence relation between a contrast between the blood and a background tissue, a first time taken from the first RF pulse to the data acquisition sequence, and a second time taken from the completion of an imaging sequence to the start of the next imaging sequence is provided. The program causes a computer to execute a first determining process to determine the first time used when the imaging sequence is repeatedly executed, based on a flow velocity of the blood, and a second determining process to determine the second time used when the imaging sequence is repeatedly executed, based on the first time determined by the first determining means and the correspondence relation.

Since the first time is determined based on the flow velocity of the blood, the blood vessel can be drawn over the entire region to be image. Further, the second time is determined based on the correspondence relation, it is possible to acquire a blood vessel image high in contrast.

Further advantages will be apparent from the following description of an exemplary embodiment as illustrated in the accompanying drawings.

### BRIEF DESCRIPTION OF THE DRAWINGS

FIG. 1 is a schematic diagram of a magnetic resonance system according to an exemplary embodiment;

FIG. 2 is an explanatory diagram used when a subject 12 is scanned;

FIG. 3 is a diagram showing a flowchart for executing the scan;

FIG. 4 is a diagram for describing a method of determining an inversion time TI;

FIG. 5 is a diagram illustrating a contrast map M1 used when determining an allowable range of a waiting time Tw;

FIG. 6 is a diagram illustrating a contrast map M2 used when determining an allowable range of a waiting time Tw;

FIG. 7 is a diagram showing a flow executed in Step ST4;

FIG. 8 is a diagram showing a concrete flowchart of Step ST40;

FIG. 9 is a diagram showing extracted map data D1;

FIG. 10 is a diagram illustrating a range v1 of r=0.5 contained in the map data D1;

FIG. 11 is a diagram illustrating a range H1 of a waiting time Tw corresponding to the range v1 of r=0.5;

FIG. 12 is a diagram showing extracted map data D2;

FIG. 13 is a diagram illustrating a range v2 of s=0.5 to 0.52 contained in the map data D2;

FIG. 14 is a diagram illustrating a range H2 of a waiting time Tw corresponding to the range v2 of s=0.5 to 0.52;

FIG. 15 is a diagram showing by comparison, the range H1 of the waiting time Tw of the contrast map M1 and the range H2 of the waiting time Tw of the contrast map M2;

FIG. 16 is a diagram illustrating a sequence PS where TI=1500 ms and Tw=2000 ms;

FIG. 17 is a diagram showing extracted map data D1;

FIG. 18 is a diagram illustrating a range v1 of r=0.5 contained in the map data D1;

FIG. 19 is a diagram illustrating a range H1 of a waiting time Tw corresponding to the range v1 of r=0.5;

FIG. 20 is a diagram showing extracted map data D2;

FIG. 21 is a diagram illustrating a range v2 of s=0.5 to 0.54 contained in the map data D2;

FIG. 22 is a diagram illustrating a range H2 of a waiting time Tw corresponding to the range v2 of s=0.5 to 0.54;

FIG. 23 is a diagram showing by comparison, the range H1 of the waiting time Tw of the contrast map M1 and the range H2 of the waiting time Tw of the contrast map M2;

FIG. 24 is a diagram illustrating a sequence PS where TI=1400 ms and Tw=2100 ms;

FIG. 25 is a diagram showing extracted map data D1;

FIG. 26 is a diagram illustrating a range H1 of a waiting time Tw corresponding to r=0.45;

FIG. 27 is a diagram showing extracted map data D2;

FIG. 28 is a diagram illustrating a range v2 of s=0.5 to 0.54 contained in the map data D2;

FIG. 29 is a diagram illustrating a range H2 of a waiting time Tw corresponding to the range v2 of s=0.5 to 0.54;

FIG. 30 is a diagram showing by comparison, the range H1 of the waiting time Tw of the contrast map M1 and the range H2 of the waiting time Tw of the contrast map M2;

FIG. 31 is a diagram illustrating a sequence PS where TI=1300 ms and Tw=2200 ms;

FIG. 32 is a diagram showing an example of an imaging sequence PS where an α° pulse is used;

FIG. 33 is a diagram showing an example in which a time Tα is set to a time between an α° pulse and a completion time point of a data acquisition sequence DAQ; and

FIG. 34 is a diagram showing an example in which a time Tα is set to a time between an α° pulse and a time point tm halfway of execution of a data acquisition sequence DAQ.

### DETAILED DESCRIPTION

An exemplary embodiment will be described hereinafter. The disclosure is however not limited to or by the following exemplary embodiments.

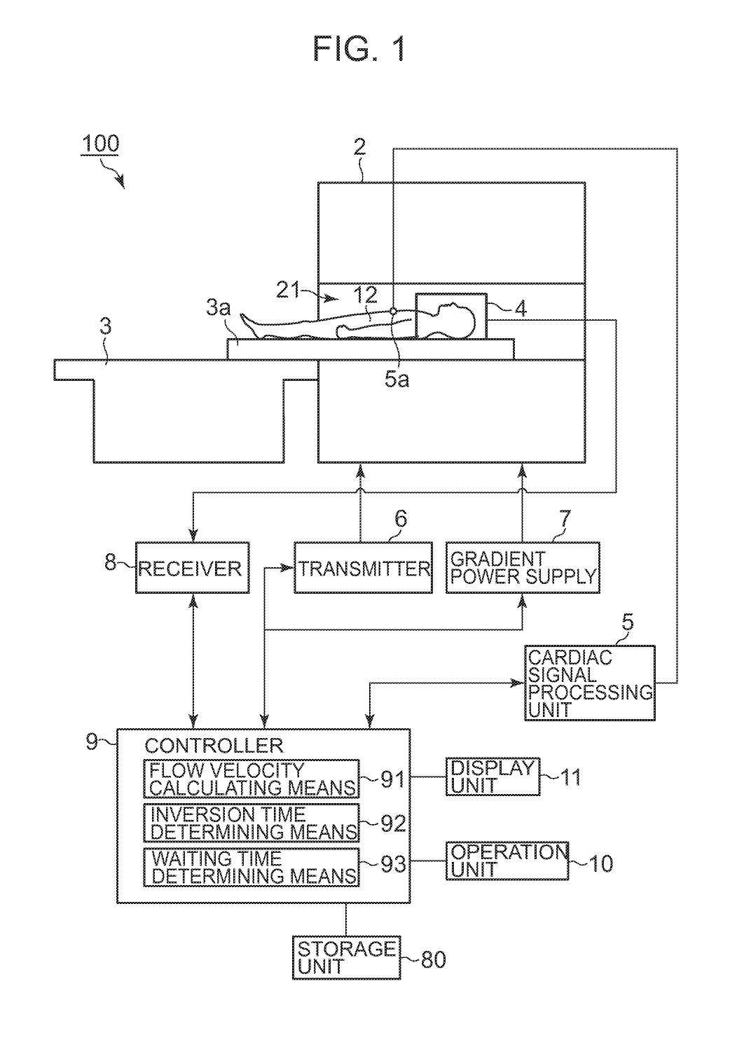

FIG. 1 is a schematic diagram of a magnetic resonance system according to an exemplary embodiment.

The magnetic resonance system (hereinafter called “MR system” where MR: Magnetic Resonance) 100 has a magnet 2, a table 3, a receiver coil 4, etc.

The magnet 2 has a bore 21 in which a subject 12 is accommodated. Further, the magnet 2 incorporates a superconductive coil, an RF coil, a gradient coil, etc. therein.

The table 3 has a cradle 3a that supports the subject 12. The cradle 3a is configured so as to be movable into the bore 21. The subject 12 is carried in the bore 21 by the cradle 3a.

The receiver coil 4 is attached to the subject 12. The receiver coil 4 receives magnetic resonance signals from the subject 12 therein.

The MR system 100 further has a biological signal processing unit 5, a transmitter 6, a gradient power supply 7, a receiver 8, a storage unit 80, a controller 9, an operation unit 10 and a display unit 11, etc.

The cardiac signal processing unit 5 receives a signal from a sensor 5a attached to the subject 12 to determine a heart or cardiac rate of the subject and an RR interval.

The transmitter 6 supplies current to the RF coil. The gradient power supply 7 supplies current to the gradient coil. The receiver 8 performs signal processing such as detection on a signal received from the receiver coil 4.

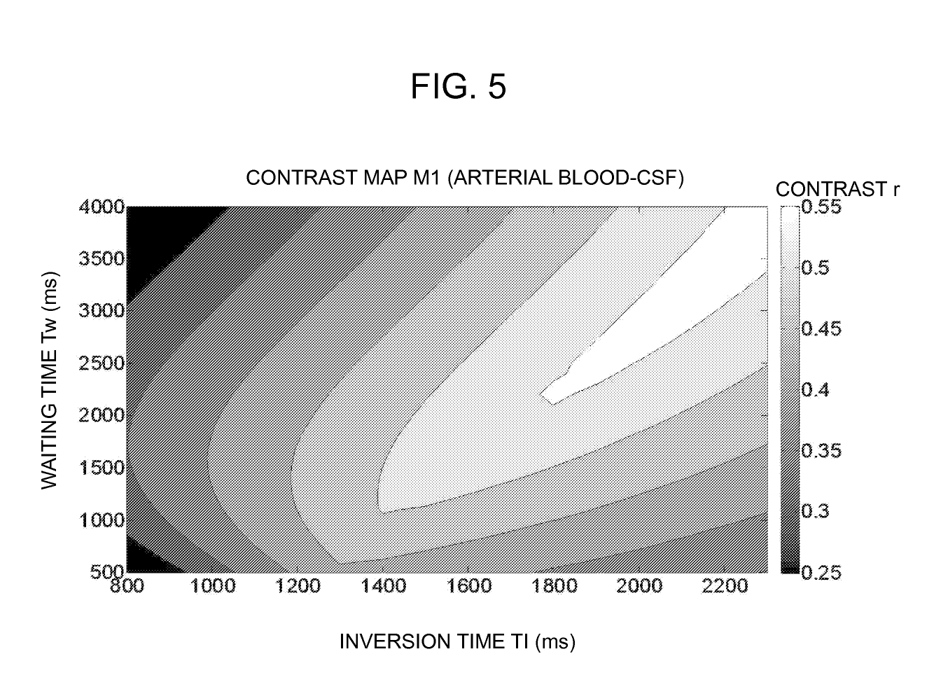

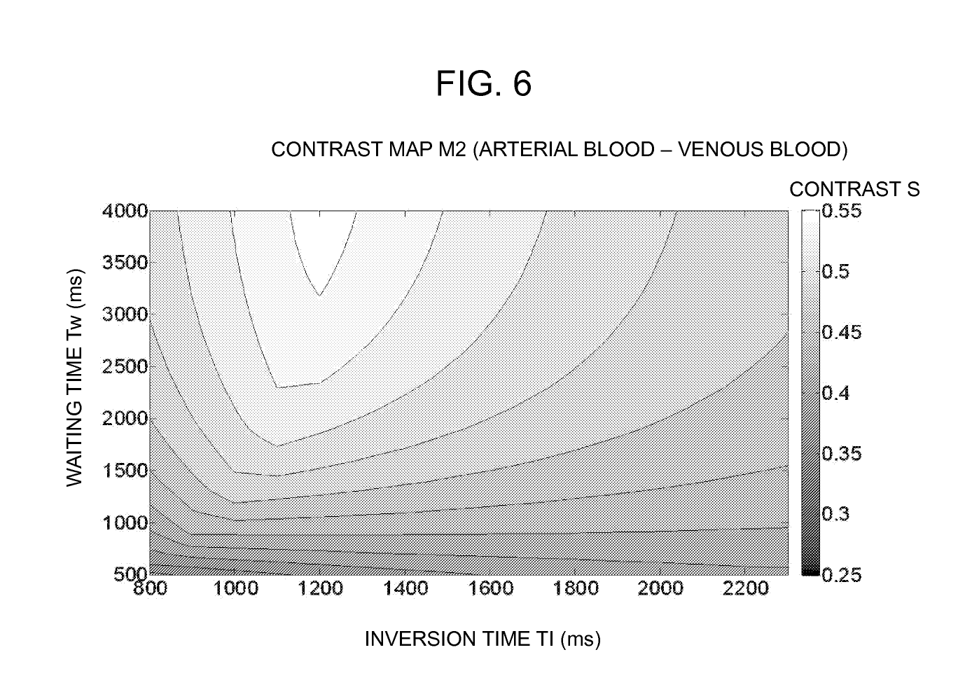

The storage unit 80 stores contrast maps M1 and M2 therein (refer to FIGS. 5 and 6). The contrast maps M1 and M2 will be described later.

The controller 9 controls the operations of respective parts of the MR system 100 so as to realize various operations of the MR system 100 such as transmission of information necessary for the display unit 11, reconstruction of an image on the basis of data received from the receiver 8, etc. The controller 9 has flow velocity calculating means 91, inversion time determining means 92 and waiting time determining means 93, etc.

The flow velocity calculating means 91 determines a flow velocity v of arterial blood, based on flow velocity information acquired by a scan.

The inversion time determining means 92 determines an inversion time TI at execution of an imaging sequence, based on the flow velocity v of the arterial blood.

The waiting time determining means 93 determines a waiting time Tw at the execution of the imaging sequence PS, on the basis of the inversion time TI determined by the inversion time determining means 92 and the contrast maps MI and M2 (refer to FIGS. 5 and 6).

Incidentally, the controller 9 is an example that configures the flow velocity calculating means 91, the inversion time determining means 92 and the waiting time determining means 93. The controller 9 functions as these means by executing a prescribed program.

The operation unit 10 is operated by an operator and inputs various information to the controller 9. The display unit 11 displays the various information thereon.

The MR system 100 is configured in the above-described manner.

The subject 12 is imaged by using the MR system 100 configured in the above-described manner.

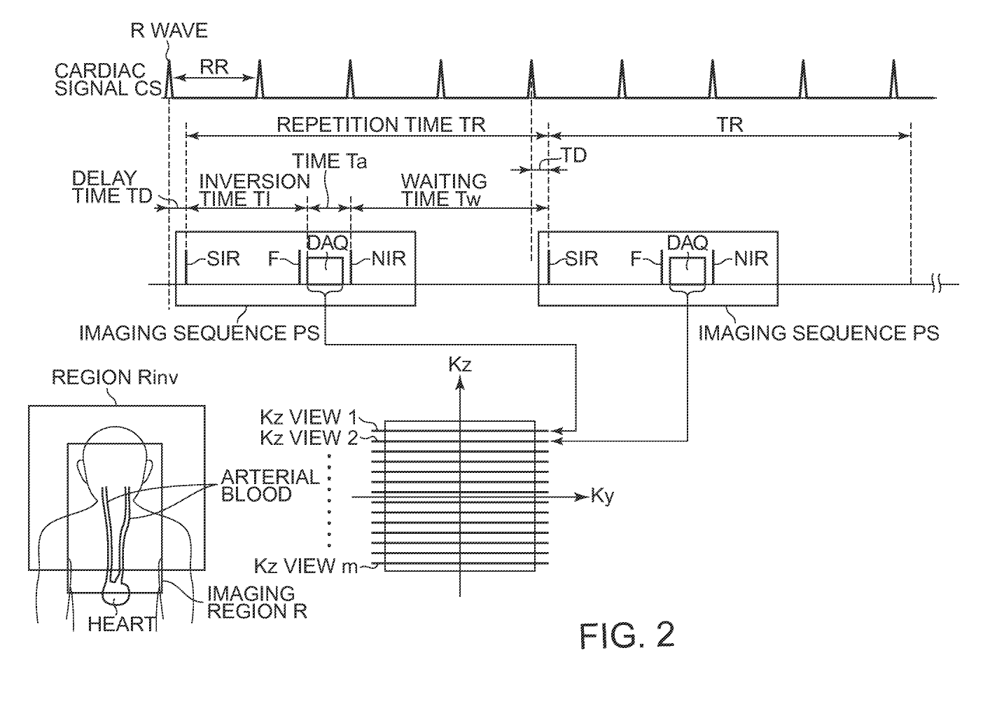

FIG. 2 is an explanatory diagram used when the subject 12 is scanned.

The upper side of FIG. 2A shows cardiac signal CS of the subject and an imaging sequence PS used when scanning the subject. The lower side of FIG. 2 shows an imaging region R of the subject and a k-space (ky-kz plane).

In the exemplary embodiment, the imaging sequence PS for depicting the arterial blood flowing through the head and neck is repeatedly executed.

Each imaging sequence PS has a selective inversion pulse SIR (Selective Inversion Recovery), a fat suppression pulse F, a data acquisition sequence DAQ, and a nonselective inversion pulse NIR.

The selective inversion pulse SIR is an RF pulse for inverting vertical magnetization of a tissue (arterial blood, venous blood, fat, muscle or the like) in a region Rinv containing the head and neck of the subject 12. The selective inversion pulse SIR is applied when a delay time TD has elapsed from an R wave of the cardiac signal CS.

When the inversion time TI has elapsed from the selective inversion pulse SIR, the data acquisition sequence DAQ for acquiring data in the imaging region R is executed. The data acquisition sequence DAQ is for example, a 3D FSE (Fast Spin Echo) or FIESTA (Fat Imaging Employing Steady state Acquisition). Since the vertical magnetization of each tissue in the region Rinv is inverted by the selective inversion pulse SIR, the vertical magnetization of each tissue in the region Rinv approaches a null point during the inversion time TI. On the other hand, since the heart is located outside the region Rinv, the vertical magnetization M of the arterial blood in the heart keeps M=1 even if the selective inversion pulse SIR is applied. Accordingly, the arterial blood of the vertical magnetization M=1 flows into the neck and head from the heart during the inversion time TI. Thus, since the data acquisition sequence DAQ is executed after the arterial blood of the vertical magnetization M=1 has flown into the neck and head during the inversion time TI, an MR image can be obtained in which the arterial blood is emphasized and depicted and the background tissue (venous blood or the like) is suppressed.

Also, the fat suppression pulse F is applied immediately before the data acquisition sequence DAQ. It is thus possible to effectively suppress a fat signal in the imaging region R. Incidentally, the fat suppression pulse F is for example, SPECIR (Spectrally Selected IR) or STIR (Short-TI IR).

Further, the nonselective inversion pulse NIR is applied immediately after the data acquisition sequence DAQ. The nonselective inversion pulse NIR is a pulse applied to invert the magnetization of each tissue in the subject.

When the waiting time Tw has elapsed after the application of the nonselective inversion pulse NIR, the next imaging sequence PS is executed.

Even in the case of the next imaging sequence PS, after a selective inversion pulse SIR and a fat suppression signal F are applied, a data acquisition sequence DAQ is executed and a nonselective inversion pulse NIR is applied. Subsequently, in the same manner as above, each imaging sequence PS is repeatedly executed. In the exemplary embodiment, data of one kz view in the ky-kz plane is acquired in one imaging sequence PS. Thus, data of all kz views 1 to m in the ky-kz plane can be acquired by executing m imaging sequences PS.

The repetition time TR will next be described.

The repetition time TR can be expressed in the following equation:

TR=TI+Ta+Tw Equation 1

where TI is inversion time,

Ta is time taken from the start of a data acquisition period DAQ to the application of a nonselective inversion pulse NIR, and

Tw is waiting time.

Further, TR is set to satisfy the following condition.

TR=RR×n Equation 2

where RR is a RR interval, and

n is an integer

In the exemplary embodiment, the value of n is a value determined depending on the inversion time TI and the waiting time Tw. A method of determining n will be described later. FIG. 2 shows n=4 as an example of n.

The above imaging sequences PS is executed to acquire the k-space data, and then the Fourier transform of the data is performed. In this way, the image of the arterial blood can be obtained. A problem however arises in that if the inversion time TI is too short, it is not possible to sufficiently obtain a blood inflow effect and depict the arterial blood with a high signal. On the other hand, a problem arises in that if the inversion time TI is too long, the background tissue is recovered and the contrast of an image is deteriorated. Further, the optimum value of the inversion time TI varies depending on a blood flow velocity and an imaging region. Accordingly, there is a need to set the inversion time TI suitable for each scan to obtain a high quality blood flow image. Since the contrast between the background tissue and the arterial blood depends even on the waiting time Tw, the waiting time Tw is also required to be set such that a satisfactory contrast is obtained. Thus, in the exemplary embodiment, the values of the inversion time TI and the waiting time Tw are determined and the imaging sequence PS is executed in such a manner that the contrast between the background tissue and the arterial blood can be increased. A description will be made below about a flowchart for determining the values of the inversion time TI and the waiting time Tw and executing the imaging sequence PS.

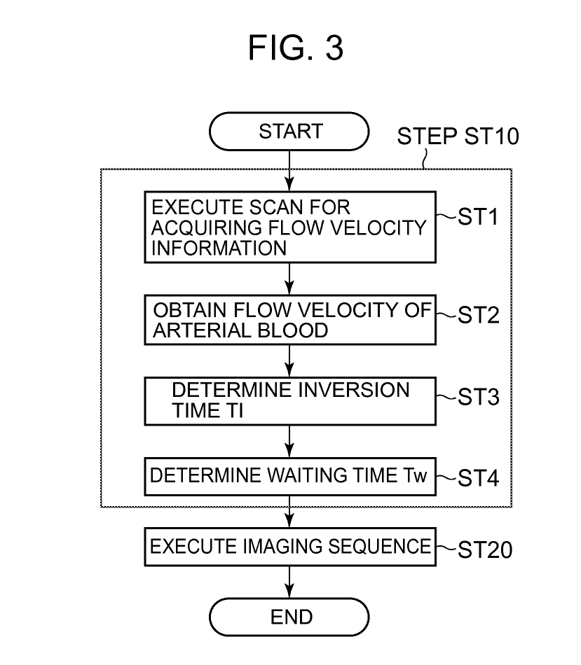

FIG. 3 is a diagram showing the flowchart for determining the values of the inversion time TI and the waiting time Tw and executing the imaging sequence PS.

The flowchart in the exemplary embodiment is roughly divided into two Steps ST10 and ST20. Step ST10 is Step for determining the values of the inversion time TI and the waiting time Tw. Step ST20 is Step for executing the imaging sequence on the basis of the inversion time TI and the waiting time Tw determined in Step ST10.

Step ST10 includes Steps ST1 to ST4. Thus, upon description of Step ST10, Steps ST1 to ST4 will be described in order.

In Step ST1, a scan for acquiring information about the flow velocity of the arterial blood of the subject is executed. The flow velocity information on the arterial blood is used for determining the inversion time TI. A concrete procedure for determining the inversion time TI using the flow velocity information on the arterial blood will be described in detail in Steps ST2 and ST3. As the scan for acquiring the flow velocity information on the arterial blood, there can be used a phase contrast MRA or the like. After this scan is executed, the flow proceeds to Step ST2.

In Step ST2, the flow velocity calculating means 91 (refer to FIG. 1) obtains the flow velocity v of the arterial blood, based on the flow velocity information acquired by the scan in Step ST1. After the flow velocity v is obtained, the flow proceeds to Step ST3.

In Step ST3, the inversion time determining means 92 (refer to FIG. 1) determines the inversion time TI, based on the flow velocity v of the arterial blood.

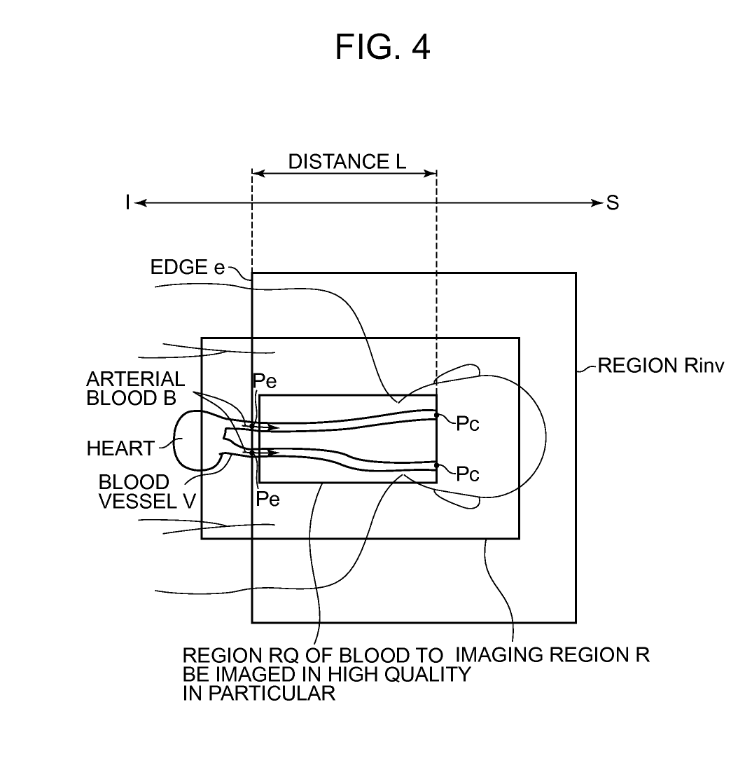

FIG. 4 is a diagram for describing a method of determining the inversion time TI.

In FIG. 4, arterial blood B that flows from the heart to the head through each blood vessel V is represented by arrows.

First consider a region RQ of blood to be imaged in high quality in particular within the imaging region R. In the exemplary embodiment, the region RQ is taken to be a region including carotid arteries.

Next, consider a time Tm required for the arterial blood B to move from a position Pe to a position Pc. The position Pe corresponds to the position of an edge e on the heart side of the region Rinv. The position Pc corresponds to the right edge position of the region RQ.

The shape of the blood vessel V between the positions Pe and Pc can be taken to approximate to a linear shape extending in an SI direction. In this case, the time Tm necessary for the arterial blood B to move from the position Pe to the position Pc can be represented by the following equation:

Tm=L/v Equation 3

where L is distance from the position Pe to Pc and

v is flow velocity of arterial blood.

It is desirable that in order to image the blood in the region RQ in high quality, the arterial blood B reaches from the position Pe to the position Pc during the time Tm. Thus, in the exemplary embodiment, the time Tm (=L/v) is taken to be the inversion time TI. Accordingly, the inversion time TI can be represented by the following equation:

TI=L/v Equation 4

Since the flow velocity v of the arterial blood in Equation 4 is obtained in Step ST2, the inversion time TI can be obtained if the value of the distance L is determined. The distance L may be determined according to, for example, the length in the SI direction of the imaging region R or may be set as a fixed value. In the present embodiment, the distance L is taken to be a value determined according to the length in the SI direction of the imaging region R. Thus, since the flow velocity v and the distance L are already known, the inversion time TI can be determined from Equation 4. Incidentally, since TI is determined by the flow velocity v and the distance L as described above (refer to Equation 4), TI can take various values according to the values of v and L. In the exemplary embodiment, consider the following three values as the value of TI for convenience of description:
TI=1500 ms
TI=1400 ms
TI=1300 ms

Then, the description of the flowchart will be continued with TI being into three cases of TI=1500 ms, TI=1400 ms and TI=1300 ms.

(1) When TI is determined to be TI=1500 ms in Step ST3:

When TI is determined to be TI=1500 ms in Step ST3, the flow proceeds to Step ST4.

In Step ST4, the waiting time determining means 93 (refer to FIG. 1) determines a waiting time Tw, based on the inversion time TI determined in Step ST3 and each contrast map. A description will be made below about each contrast map used when determining the waiting time Tw (refer to FIGS. 5 and 6).

FIGS. 5 and 6 are diagrams showing the contrast maps M1 and M2 each used when determining the waiting time Tw.

The contrast map M1 (refer to FIG. 5) represents a contrast r between the arterial blood and CSF (cerebrospinal fluid). This map is prepared before scanning the subject. The horizontal axis of the contrast map M1 indicates the inversion time TI, and the vertical axis thereof indicates the waiting time Tw. The value of the contrast r is associated with the combination of the value of the inversion time TI and the value of the waiting time Tw. The value of the contrast r can be determined by, for example, calculating a signal value of the arterial blood and a signal value of CSF using the Bloch equation and taking the ratio between these signal values.

The contrast r has a value in a range of 0.25≦r≦0.55. In the contrast map M1, the differences in the value between the contrasts r are represented with brightness/darkness of gray. r=0.25 corresponds to black, and r=0.55 corresponds to white. As the contrast r becomes larger from 0.25, the color of the contrast map M1 gradually approaches from black to white.

On the other hand, the contrast map M2 (refer to FIG. 6) represents a contrast s between the arterial blood and the venous blood. This map is prepared before scanning the subject. The horizontal axis of the contrast map M2 indicates the inversion time TI, and the vertical axis thereof indicates the waiting time Tw. The value of the contrast s is associated with the combination of the value of the inversion time TI and the value of the waiting time Tw. The value of the contrast s can be determined by, for example, calculating a signal value of the arterial blood and a signal value of the venous blood using the Bloch equation and taking the ratio between these signal values.

The contrast s has a value in a range of 0.25≦s≦0.55. In the contrast map M2, the differences in the value between the contrasts s are represented with brightness/darkness of gray. s=0.25 corresponds to black, and s=0.55 corresponds to white. As the contrast s becomes larger from 0.25, the color of the contrast map M2 gradually approaches from black to white.

In Step ST4, the waiting time Tw is determined using the contrast maps M1 and M2 shown in FIGS. 5 and 6. A description will be made below about the operation of Step ST4.

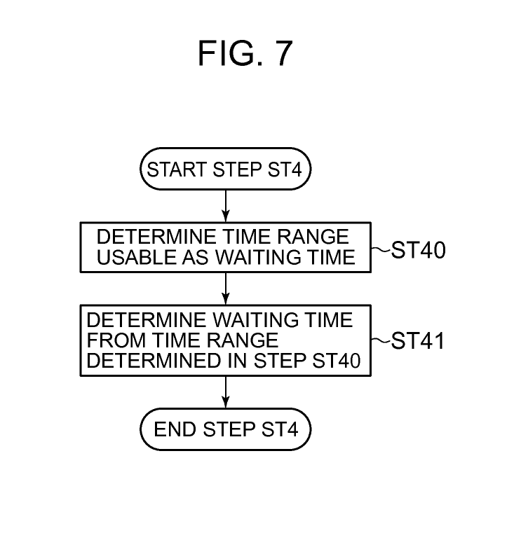

FIG. 7 is a diagram showing a flow executed in Step ST4.

Step ST4 is divided in to two Steps ST40 and ST41.

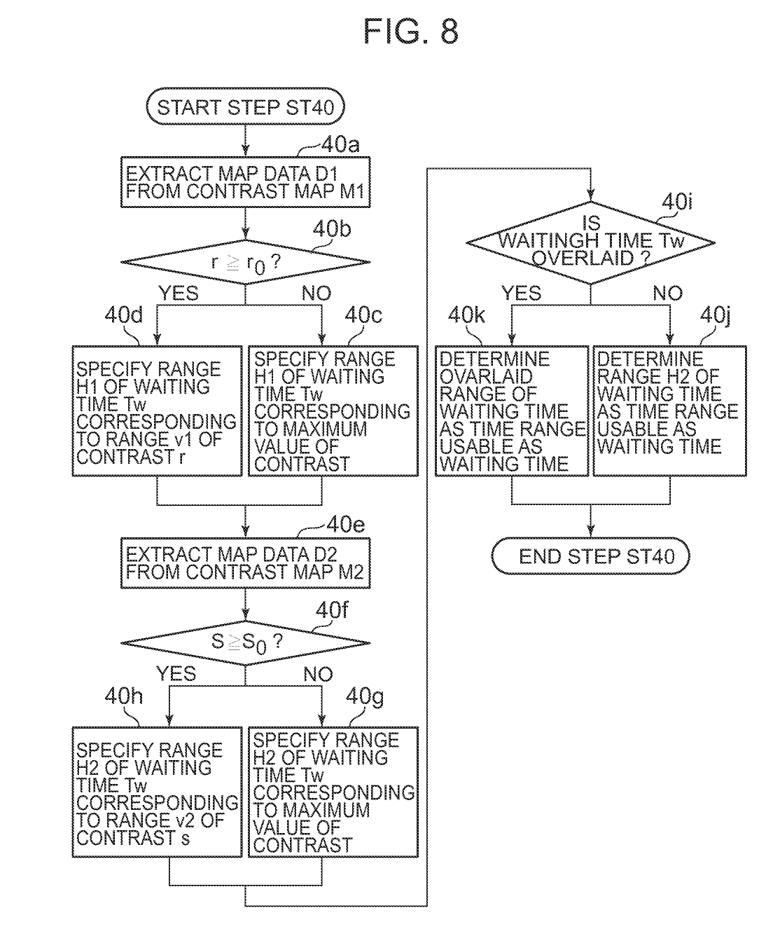

In Step ST40, a process for determining a time range usable as the waiting time Tw is executed. A concrete flowchart of Step ST40 is shown in FIG. 8. FIG. 8 will be described later.

In Step ST41, a process is executed for determining a waiting time Tw at the execution of the sequence from the time range determined in Step ST40.

A description will be made below about Steps ST40 and ST41 of Step ST4.

In Step ST4, Step ST40 is first executed (refer to FIG. 8).

FIG. 8 is a diagram showing a detailed flowchart of Step ST40.

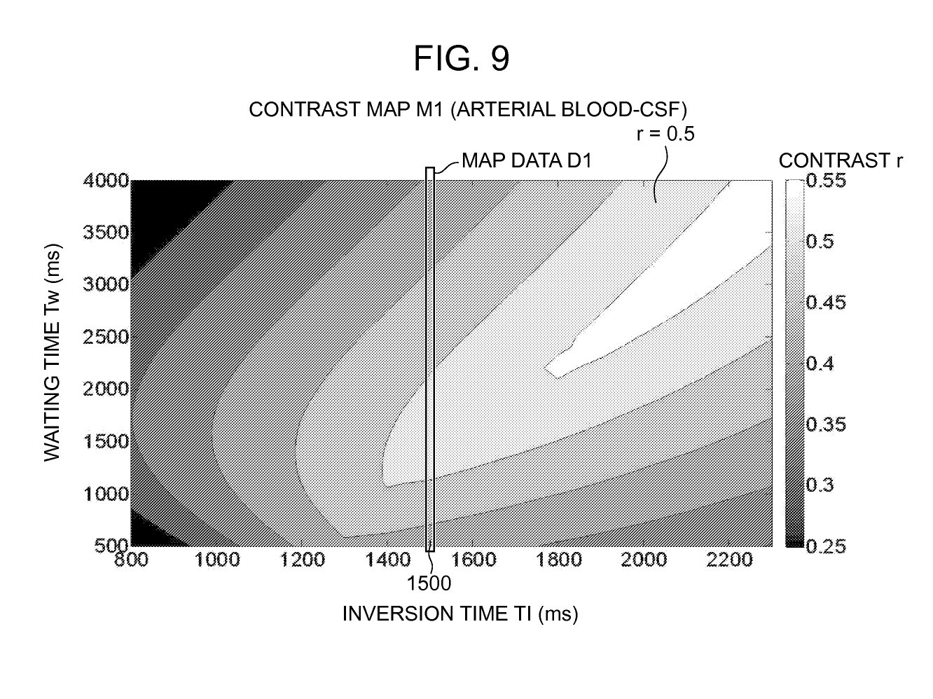

In Step 40a, the waiting time determining means 93 extracts map data at the inversion time TI=1500 (ms) determined in Step ST3 from the contrast map M1 (refer to FIG. 5). The extracted map data D1 is shown in FIG. 9. After the extraction of the map data D1, the flow proceeds to Step 40b.

In Step 40b, the waiting time determining means 93 determines whether or not the contrast r that satisfies the following condition is contained in the map data D1:

r≧r₀ Equation 5

where r₀ is the lower limit value allowable as the value of the contrast r between the arterial blood and CSF.

In the exemplary embodiment, r₀ is taken to be set r₀=0.5. In this case, Equation 5 is represented by the following equation:

r≧0.5 Equation 6

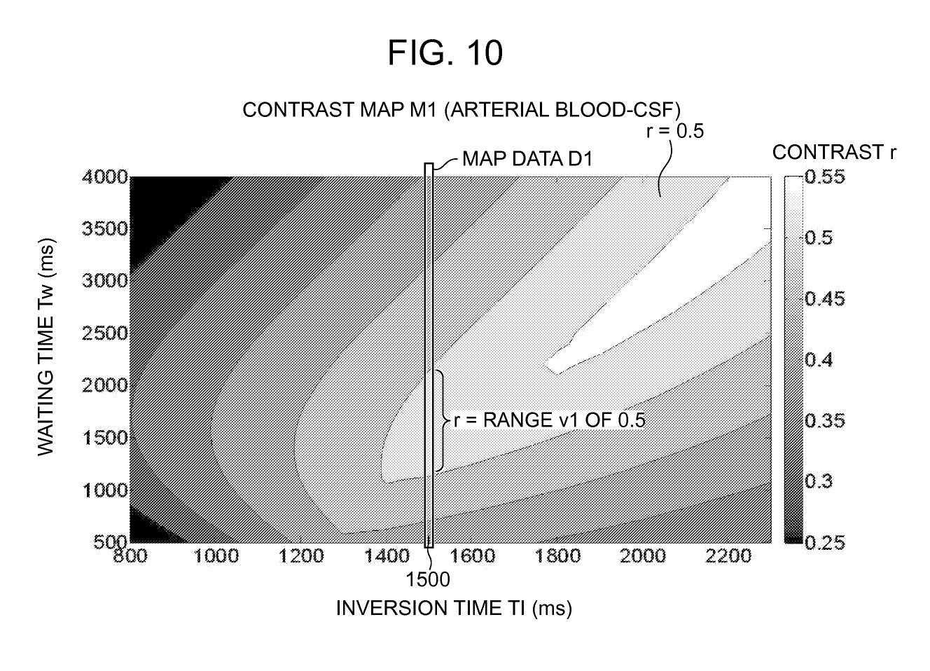

Thus, the waiting time determining means 93 judges whether or not the contrast r that satisfies the Equation 6 is contained in the map data D1 at the waiting time TI=1500 (ms). When the contrast r that satisfies the Equation 6 is contained in the map data D1, the flow proceeds to Step 40d. On the other hand, when the contrast r that satisfies the Equation 6 is not contained in the map data D1, the flow proceeds to Step 40c. Referring to FIG. 9, it is understood that the map data D1 crosses a region of r=0.5. Thus, the contrast r that satisfies the Equation 6 is contained in the map data D1. FIG. 10 shows a range v1 of r=0.5 contained in the map data D1. Since the contrast r that satisfies the Equation 6 is contained in the map data D1, the flow proceeds to Step 40d.

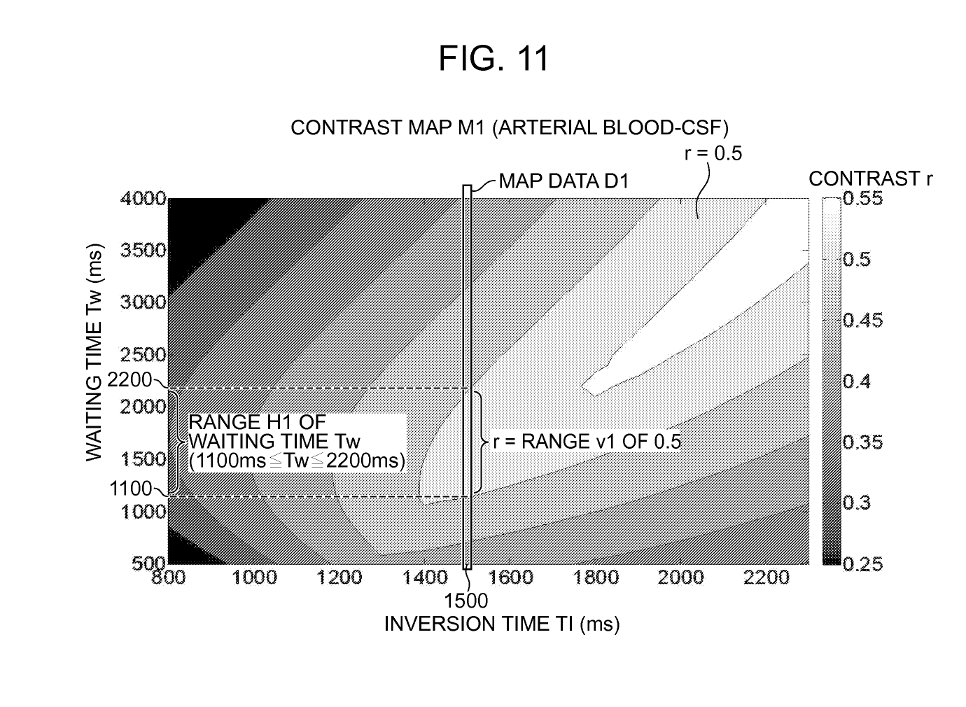

In Step 40d, the waiting time determining means 93 specifies the range of a waiting time Tw corresponding to the range v1 of r=0.5. FIG. 11 shows the range H1 of the waiting time Tw corresponding to the range v1 of r=0.5. The range H1 of the waiting time Tw is taken to be the following range herein:

1100 (ms)≦Tw≦2200 (ms) Equation 7

After the range H1 of the waiting time Tw in the contrast map M1 is obtained, the flow proceeds to Step 40e.

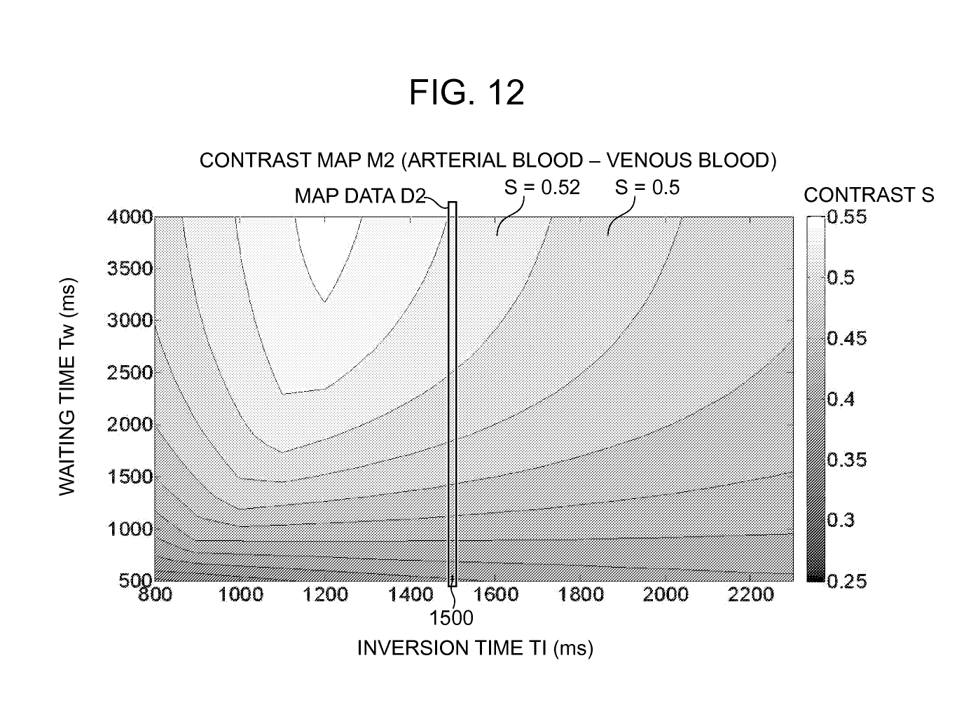

In Step 40e, the waiting time determining means 93 extracts map data at the inversion time TI=1500 (ms) determined in Step ST3 from the contrast map M2 (refer to FIG. 6). The extracted map data D2 is shown in FIG. 12. After the extraction of the map data D2, the flow proceeds to Step 40f.

In Step 40f, the waiting time determining means 93 determines whether or not the contrast s that satisfies the following condition is contained in the map data D2:

s≧s₀ Equation 8

where s₀ is the lower limit value allowable as the value of the contrast s between the arterial blood and the venous blood.

In the exemplary embodiment, s₀ is taken to be set to s₀=0.5. In this case, Equation 8 is represented by the following equation:

s≧0.5 Equation 9

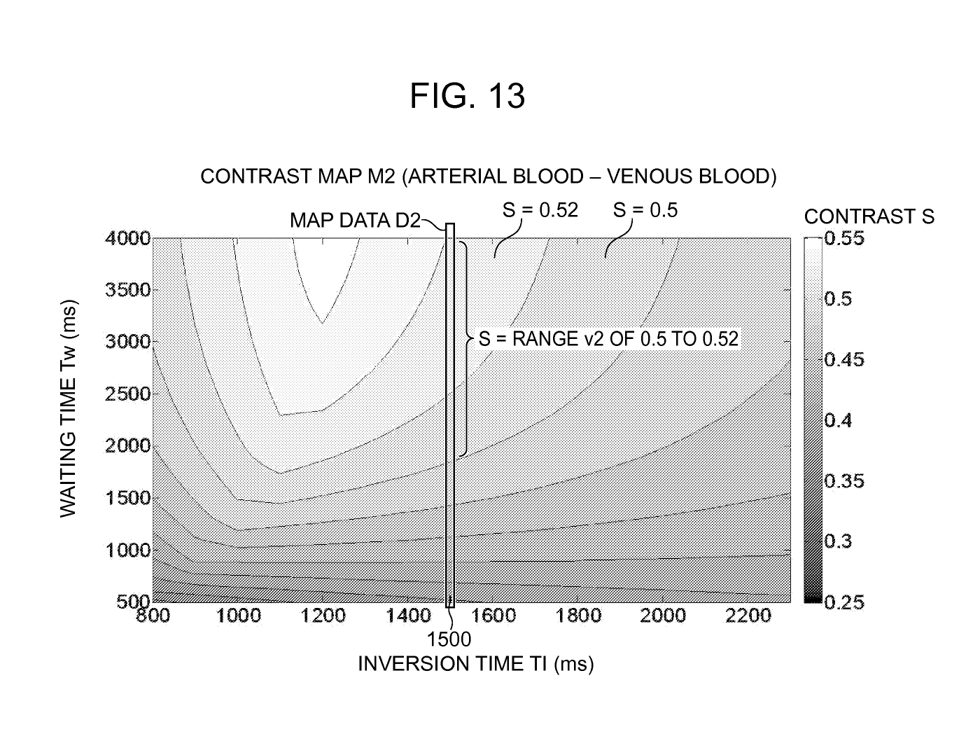

Thus, the waiting time determining means 93 judges whether or not the contrast s that satisfies Equation 9 is contained in the map data D2 at the inversion time TI=1500 (ms). When the contrast s that satisfies Equation 9 is contained in the map data D2, the flow proceeds to Step 40h. On the other hand, when the contrast s that satisfies Equation 9 is not contained in the map data D2, the flow proceeds to Step 40g. Referring to FIG. 12, it is understood that the map data D2 crosses regions of contrasts 0.5 to 0.52. Thus, the contrast s that satisfies Equation 9 is contained in the map data D2. FIG. 13 shows a range v2 of s=0.5 to 0.52 contained in the map data D2. Since the contrast s that satisfies the Equation 9 is contained in the map data D2, the flow proceeds to Step 40h.

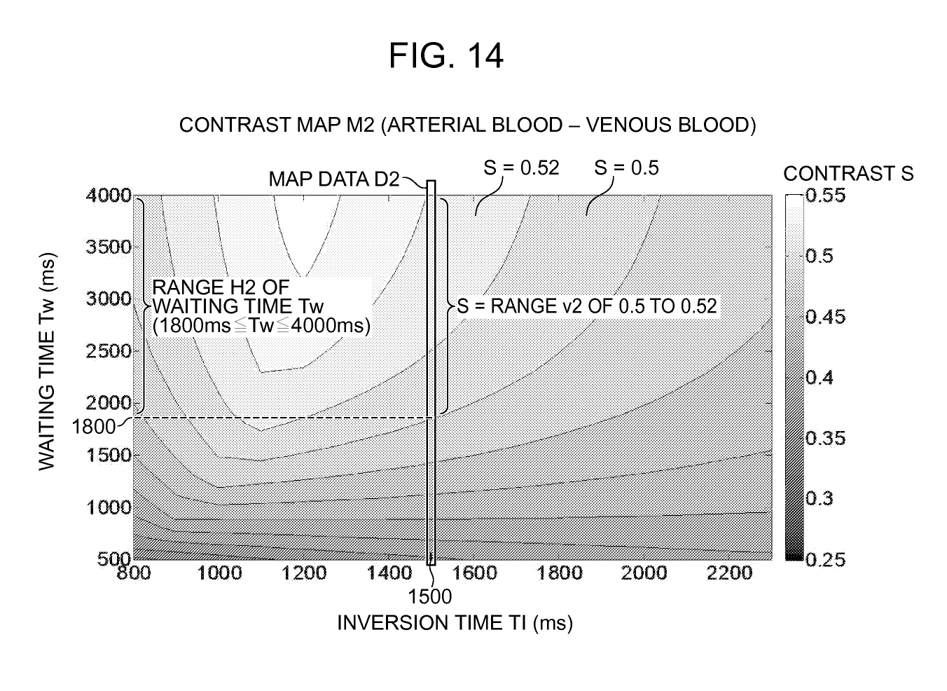

In Step 40h, the waiting time determining means 93 specifies a range H2 of a waiting time Tw corresponding to the range v2 of s=0.5 to 0.52. FIG. 14 shows the range H2 of the waiting time Tw corresponding to the range v2 of s=0.5 to 0.52. The range H2 of the waiting time Tw is taken to be the following range herein:

1800 (ms)≦Tw≦4000 (ms) Equation 10

After the range H2 of the waiting time Tw in the contrast map M2 is obtained, the flow proceeds to Step 40i.

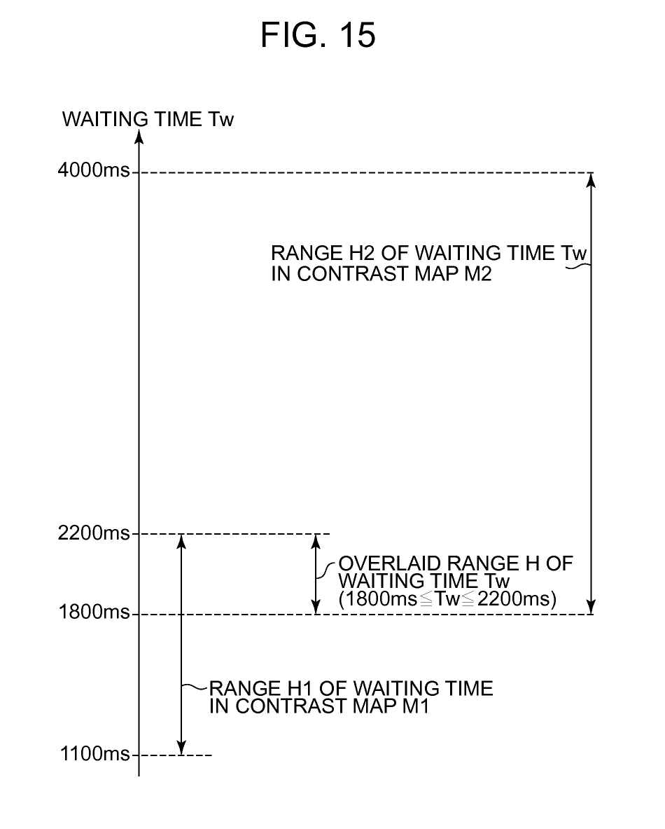

In Step 40i, the waiting time determining means 93 judges whether or not an overlaid part exists between the range H1 (refer to FIG. 11) of the waiting time Tw in the contrast map M1 and the range H2 (refer to FIG. 14) of the waiting time Tw in the contrast map M2. The range H1 of the waiting time Tw in the contrast map M1 and the range H2 of the waiting time Tw in the contrast map M2 are shown in FIG. 15 by comparison.

When the overlaid range H of the waiting time Tw exists between the ranges H1 and H2, the flow proceeds to Step 40k. On the other hand, when the overlaid range H does not exist therebetween, the flow proceeds to Step 40j. In FIG. 15, the waiting time Tw is overlaid therebetween within the range of 1800 ms≦Tw≦2200 ms. Therefore, the flow proceeds to Step 40k.

In Step 40k, the waiting time determining means 93 determines the overlaid range H of the waiting time Tw to be a time range usable as the waiting time Tw. Here, the waiting time Tw is overlaid within the range of 1800 ms≦Tw≦2200 ms. Thus, the time range H usable as the waiting time Tw is represented as the following range:

1800 ms≦Tw≦2200 ms Equation 11

After the time range usable as the waiting time Tw is determined, the flow of Step ST40 is completed. After the completion of Step ST40, the flow proceeds to Step ST41 (refer to FIG. 7).

In Step ST41, the waiting time determining means 93 determines a waiting time Tw at the execution of the imaging sequence PS from the time range H usable as the waiting time Tw. A method of determining the waiting time Tw will be described below.

From Equation 1, the waiting time Tw can be represented by the following equation:

Tw=TR−(TI+Ta) Equation 12

Substituting Equation 2 into Equation 12 yields the following equation:

Tw=RR×n×−(TI+Ta) Equation 13

In the Equation 13, RR is a value that can be calculated from a cardiac signal. RR is taken to be RR=1000 ms herein. Further, the time Ta is a time taken from the starting point of time of the data acquisition sequence DAQ to the time of application of the nonselective inversion pulse NIR. Since the time Ta is a fixed value determined by the imaging sequence PS, the time Ta is a known value. Here, Ta is taken to be Ta=500 ms. Thus, Equation 13 is represented by the following equation:

Tw=1000n−(TI+500) Equation 14

Further, the inversion time TI is calculated as TI=1500 ms in Step ST3. Thus, substituting TI=1500 ms into Equation 14 yields the following equation:

$$\begin{matrix}Tw = 1000 n - ( TI + 500 ) \\ = 1000 n - ( 1500 + 500 ) \\ = 1000 n - 2000\end{matrix} \qquad Equation 15$$

In Step ST40, the time range H usable as the waiting time Tw is obtained as 1800 ms≦Tw≦2200 ms (refer to Equation 11). Thus, the waiting time Tw represented by the equation (15) is needed to satisfy the following equation:

1800 ms≦Tw=1000n−2000≦2200 ms Equation 16

The waiting time determining means 93 determines n that satisfies Equation 16. n that satisfies Equation 16 is n=4. After n=4 is obtained, n=4 is substituted into Equation 15. By substituting n=4 into Equation 15, the waiting time Tw can be obtained as follows:

$$\begin{matrix}Tw = 1000 n - 2000 \\ = 1000 \times 4 - 2000 \\ = 2000 ms\end{matrix}$$

Accordingly, the waiting time can be determined as Tw=2000 ms.

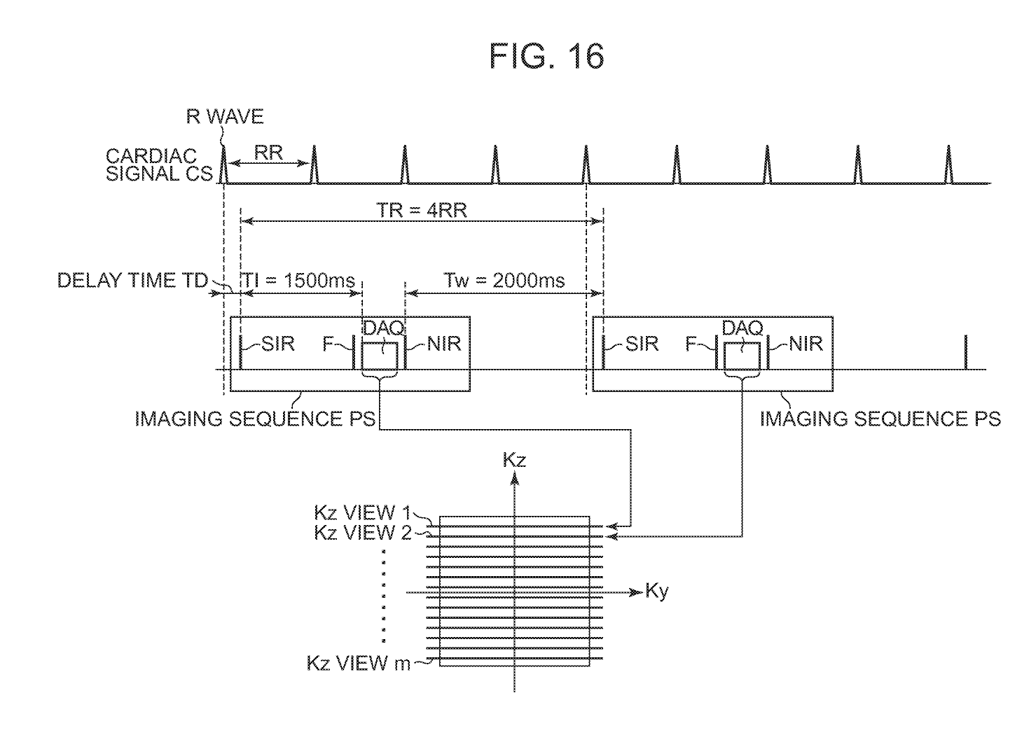

As described above, TI=1500 ms and Tw=2000 ms are obtained by executing the processes of Steps ST3 and ST4. FIG. 16 shows an imaging sequence PS where TI=1500 ms and Tw=2000 ms. Incidentally, since n=4, substituting n=4 into Equation 2 yields TR=4RR.

After the waiting time Tw is determined, the flow proceeds to Step ST20 (refer to FIG. 3).

In Step ST20, the k-space data is acquired in accordance with the imaging sequence PS shown in FIG. 16, and the flowchart is terminated.

In the exemplary embodiment, the time Tm required for the arterial blood B to move from the position Pe to the position Pc is determined (refer to FIG. 4 and Equation 3), and the time Tm is set as the inversion time TI. Thus, since the data acquisition sequence DAQ is executed when the arterial blood B having sufficiently large vertical magnetization flows into the entire region RQ (refer to FIG. 4), it is possible to obtain a sufficiently large arterial blood signal from the region RQ.

Further, in the exemplary embodiment, the range H1 (refer to FIG. 11) of the waiting time Tw when the contrast r between the arterial blood and CSF becomes r≧0.5, and the range H2 (refer to FIG. 14) of the waiting time Tw when the contrast s between the arterial blood and the venous blood becomes s≧0.5 are determined based on the contrast maps M1 and M2. Then, the range H of the waiting time Tw, which is overlaid on both ranges H1 and H2, is identified. The waiting time Tw at the execution of the imaging sequence PS is determined from within the range H. Thus, since it is possible for the contrast r between the arterial blood and CSF to have a sufficiently large value and is possible for the contrast s between the arterial blood and the venous blood to have a sufficiently large value, a blood vessel image having a satisfactory contrast can be obtained.

Incidentally, although the waiting time Tw is defined using the RR interval in Equation 13, the waiting time Tw may be defined using the cardiac rate instead of the RR interval. If the cardiac rate is represented by BPM, the relation of RR=(60/BPM)×103 (ms) is established between the RR interval and the cardiac rate. Therefore, the waiting time Tw can be determined even if the cardiac rate BPM is calculated instead of the RR interval.

The above description has shown the example in which the inversion time TI is determined as TI=1500 ms in Step ST3. A description will next be made about the case where TI=1400 ms.

(2) When TI is determined as TI=1400 ms in Step ST3:

When TI is determined to be TI=1400 ms in Step ST3, the flow proceeds to Step 40a (refer to FIG. 8) as with the case where TI=1500 ms.

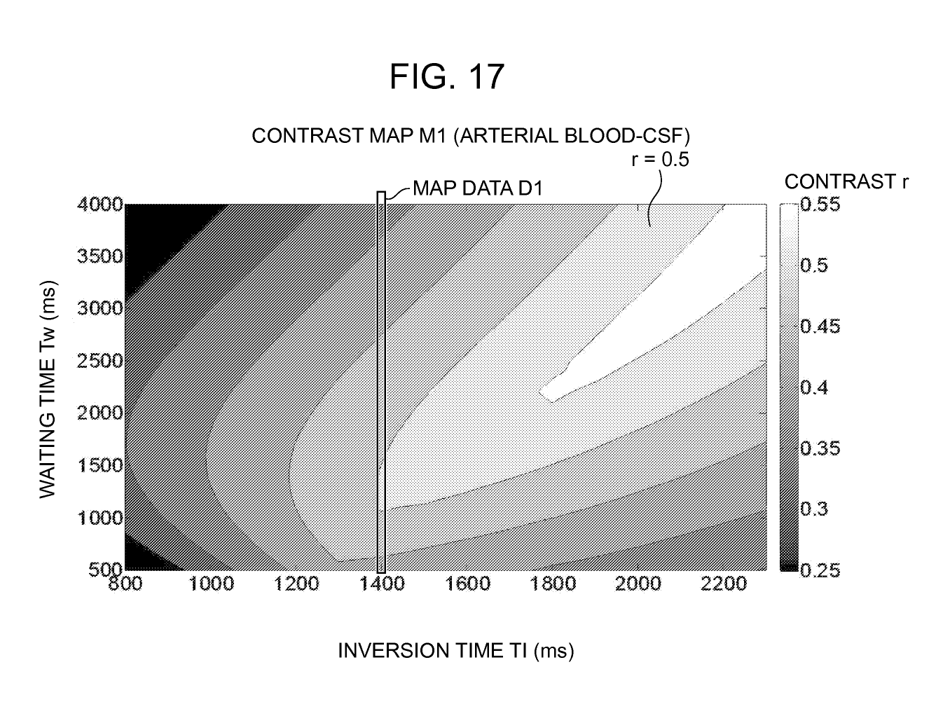

In Step 40a, the waiting time determining means 93 extracts map data at the inversion time TI=1400 (ms) determined in Step ST3 from the contrast map M1 (refer to FIG. 5). The extracted map data D1 is shown in FIG. 17. After the extraction of the map data D1, the flow proceeds to Step 40b.

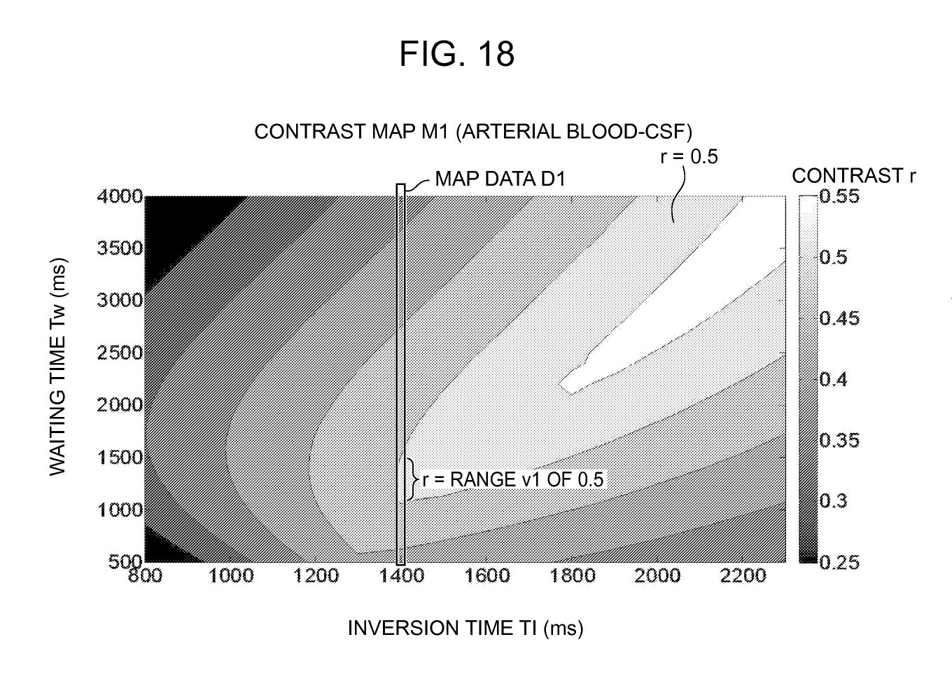

In Step 40b, the waiting time determining means 93 judges whether or not the contrast r that satisfies r≧0.5 (refer to Equation 6) is contained within the map data D1. Referring to FIG. 17, it is understood that the map data D1 slightly crosses a region of a contrast 0.5. Thus, the contrast r that satisfies r≧0.5 is contained in the map data D1. FIG. 18 shows a range v1 of r=0.5 contained in the map data D1. Since the contrast r that satisfies r≧0.5 is contained in the map data D1, the flow proceeds to Step 40d.

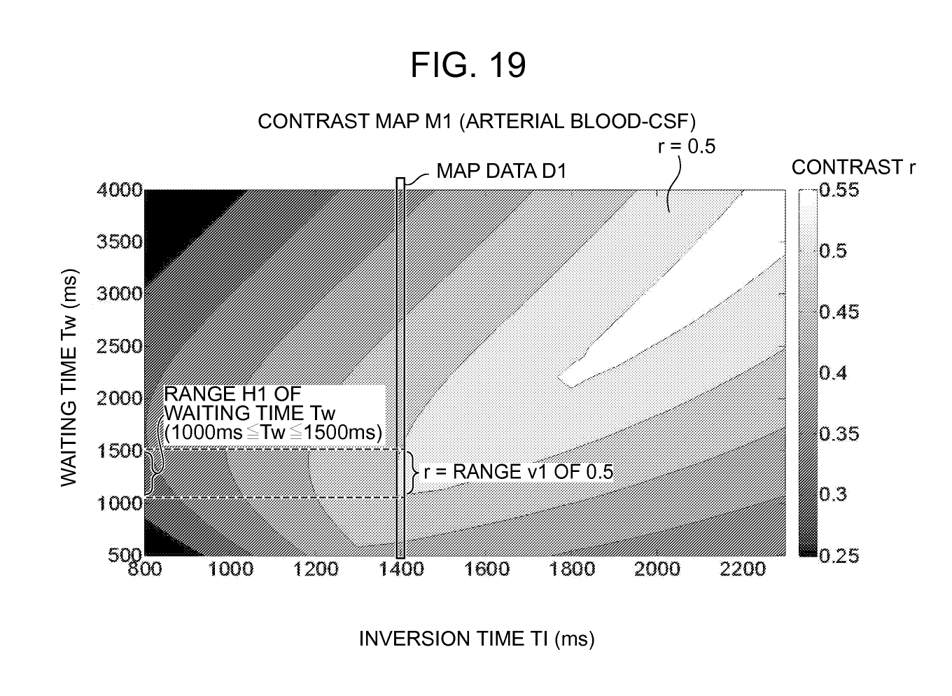

In Step 40d, the waiting time determining means 93 specifies a range of a waiting time Tw corresponding to the range v1 of r=0.5. FIG. 19 shows the range H1 of the waiting time Tw corresponding to the range v1 of r=0.5. The range H1 of the waiting time Tw is taken to be the following range herein:

1000 (ms)≦Tw≦1500 (ms) Equation 17

After the range H1 of the waiting time Tw in the contrast map M1 is obtained, the flow proceeds to Step 40e.

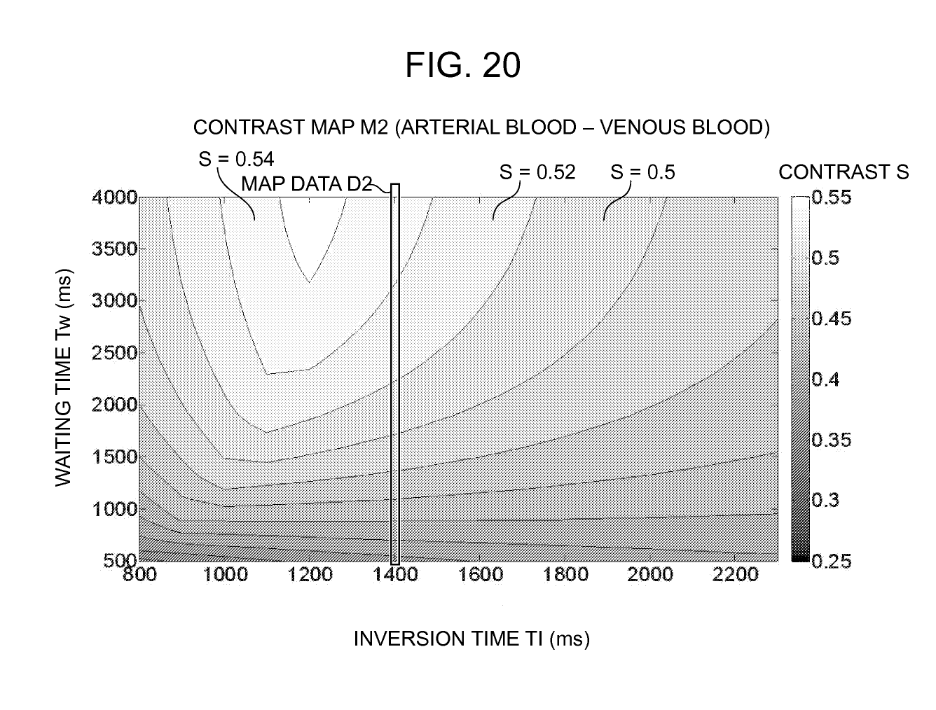

In Step 40e, the waiting time determining means 93 extracts map data at the inversion time TI=1400 (ms) determined in Step ST3 from the contrast map M2 (refer to FIG. 6). The extracted map data D2 is shown in FIG. 20. After the extraction of the map data D2, the flow proceeds to Step 40f.

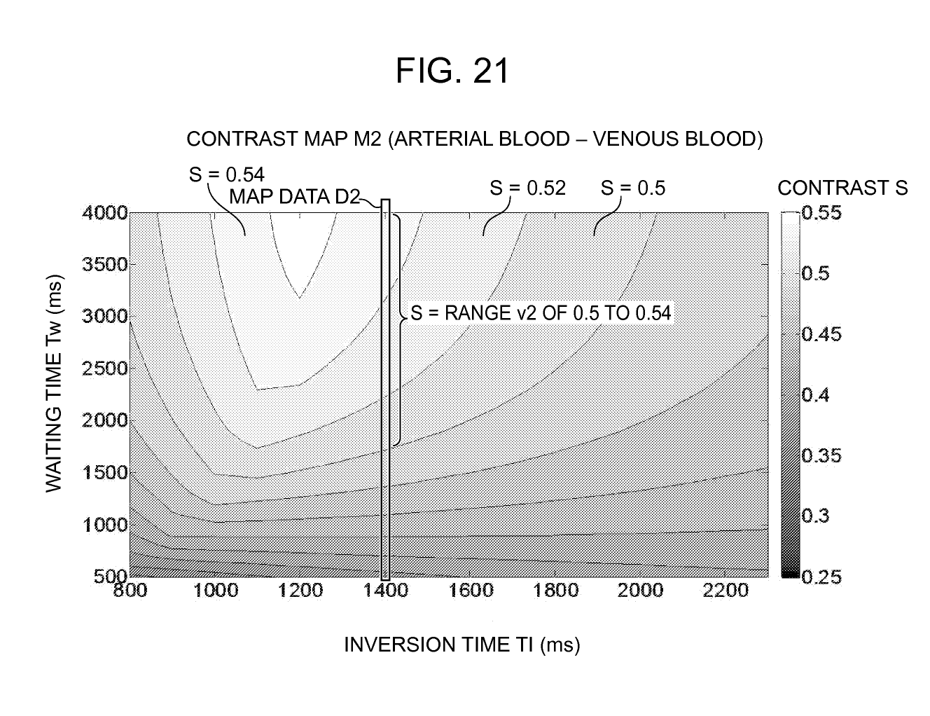

In Step 40f, the waiting time determining means 93 judges whether or not the contrast s that satisfies s≧0.5 (refer to Equation 9) is contained in the map data D1. Referring to FIG. 20, it is understood that the map data D2 crosses regions of contrasts 0.5 to 0.54. Thus, the contrast s that satisfies s≧0.5 is contained in the map data D2. FIG. 21 shows a range v2 of s=0.5 to 0.54 contained in the map data D2. Since the contrast s that satisfies s≧0.5 is contained in the map data D2, the flow proceeds to Step 40h.

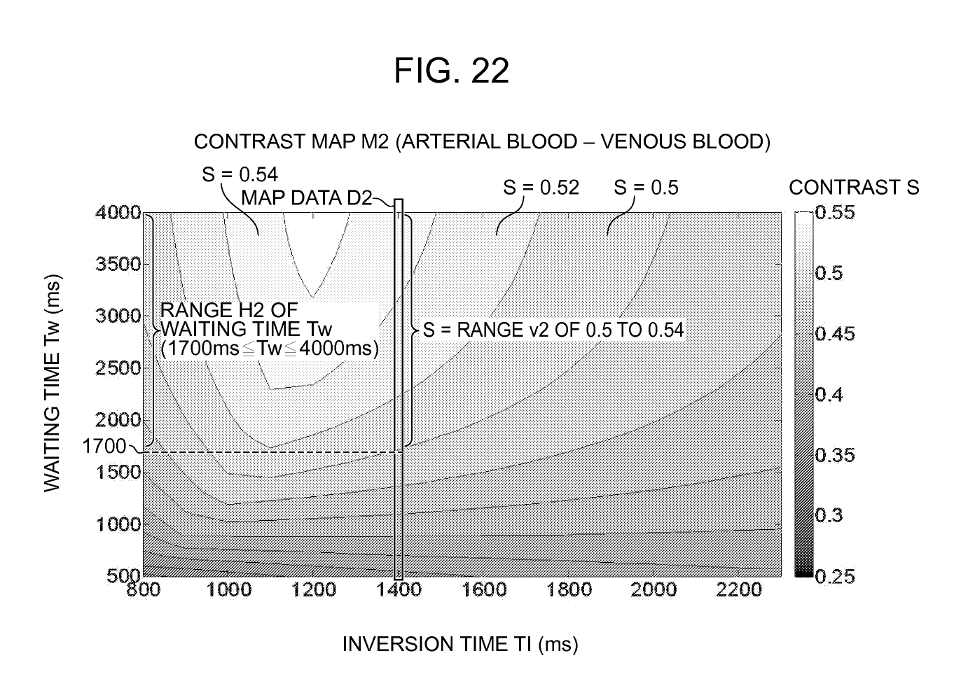

In Step 40h, the waiting time determining means 93 specifies a range H2 of a waiting time Tw corresponding to the range v2 of s=0.5 to 0.54. FIG. 22 shows the range H2 of the waiting time Tw corresponding to the range v2 of s=0.5 to 0.54. The range H2 of the waiting time Tw is taken to be the following range herein:

1700 (ms)≦Tw≦4000 (ms) Equation 18

After the range H2 of the waiting time Tw in the contrast map M2 is obtained, the flow proceeds to Step 40i.

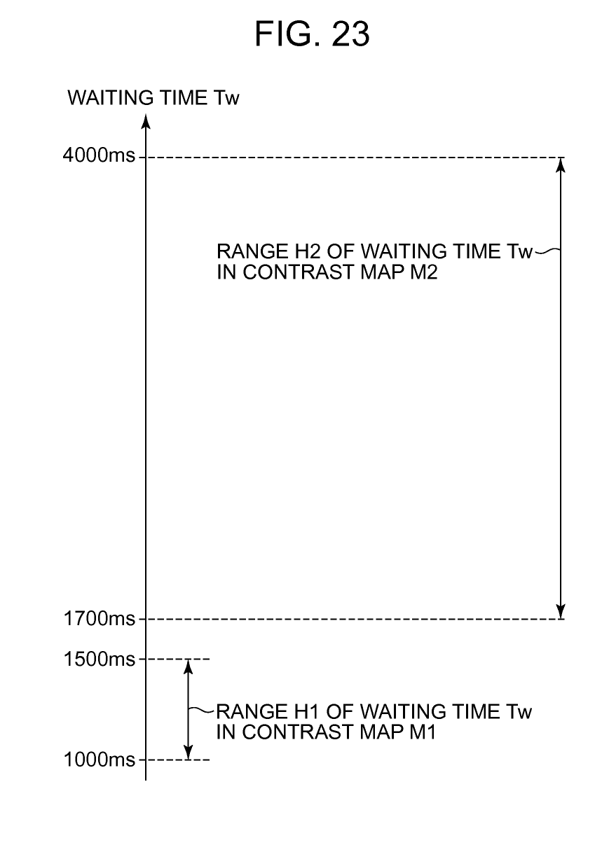

In Step 40i, the waiting time determining means 93 judges whether or not an overlaid part exists between the range H1 (refer to FIG. 19) of the waiting time Tw in the contrast map M1 and the range H2 (refer to FIG. 22) of the waiting time Tw in the contrast map M2. The range H1 of the waiting time Tw in the contrast map M1, and the range H2 of the waiting time Tw in the contrast map M2 are shown in FIG. 23 by comparison. The overlaid range of the waiting time Tw does not exist in FIG. 23. Therefore, the flow proceeds to Step 40j.

In Step 40j, the waiting time determining means 93 determines the range H2 of the waiting time Tw in the contrast map M2 as a time range H usable as a waiting time Tw. Since the range H2 of the waiting time Tw in the contrast map M2 is 1700 ms≦Tw≦4000 ms herein, the time range H usable as the waiting time Tw becomes the following range:

1700 ms≦Tw≦4000 ms Equation 19

By setting the time range H usable as the waiting time Tw to the range of Equation 19, the contrast s between the arterial blood and the venous blood can be made greater than or equal to s≧0.5. Incidentally, when the time range H usable as the waiting time Tw is set to the range of Equation 19, the contrast r between the arterial blood and CSF becomes r<0.5 (refer to FIG. 19). Therefore, it is considered that the contrast r between the arterial blood and CSF becomes small. Since, however, the position of CSF is shifted in an RL direction with respect to the position of the carotid artery important for diagnosis, it is considered not to be interferent much in diagnosing the carotid artery even if the contrast r between the arterial blood and CSF is small.

When the time range usable as the waiting time Tw is determined, the flow of Step ST40 is completed. After the completion of Step ST40, the flow proceeds to Step ST41 (refer to FIG. 7).

In Step ST41, the waiting time determining means 93 determines a waiting time Tw at the execution of an imaging sequence PS from the time range usable as the waiting time Tw. A method of determining the waiting time Tw will be described below.

When RR and Ta are taken to be RR=1000 ms and Ta=500 ms as previously described, the waiting time Tw can be represented by Equation 14. Equation 14 is shown again below.

Tw=1000n−(TI+500) Equation 14

Here, the inversion time TI has been calculated as TI=1400 ms in Step ST3. Thus, substituting TI=1400 ms into Equation 14 yields the following equation:

$$\begin{matrix}Tw = 1000 n - ( TI + 500 ) \\ = 1000 n - ( 1400 + 500 ) \\ = 1000 n - 1900\end{matrix} \qquad Equation 20$$

Further, in Step ST40, the time range H usable as the waiting time Tw has been determined as 1700 ms≦Tw≦4000 ms (refer to Equation 19). Thus, the waiting time Tw expressed in Equation 20 is required to satisfy the following equation:

1700 ms≦Tw=1000n−1900≦4000 ms Equation 21

The waiting time determining means 93 determines n that satisfies Equation 21. n that satisfies Equation 21 is n=4 and 5. Substituting n=4 into the Equation 20 yields the following value as the waiting time Tw:

$$\begin{matrix}Tw = 1000 n - 1900 \\ = 1000 \times 4 - 1900 \\ = 2100 ms\end{matrix}$$

On the other hand, when n=5 is substituted into the equation (20), the waiting time Tw becomes the following value:

$$\begin{matrix}Tw = 1000 n - 1900 \\ = 1000 \times 5 - 1900 \\ = 3100 ms\end{matrix}$$

Accordingly, the two waiting times Tw=2100 ms and Tw=3100 ms are obtained. Thus, when the waiting time Tw that satisfies Equation 21 exists in plural form, the minimum waiting time Tw (=2100 ms) is determined as the waiting time Tw at the execution of the imaging sequence PS in such a manner that a scan time becomes short.

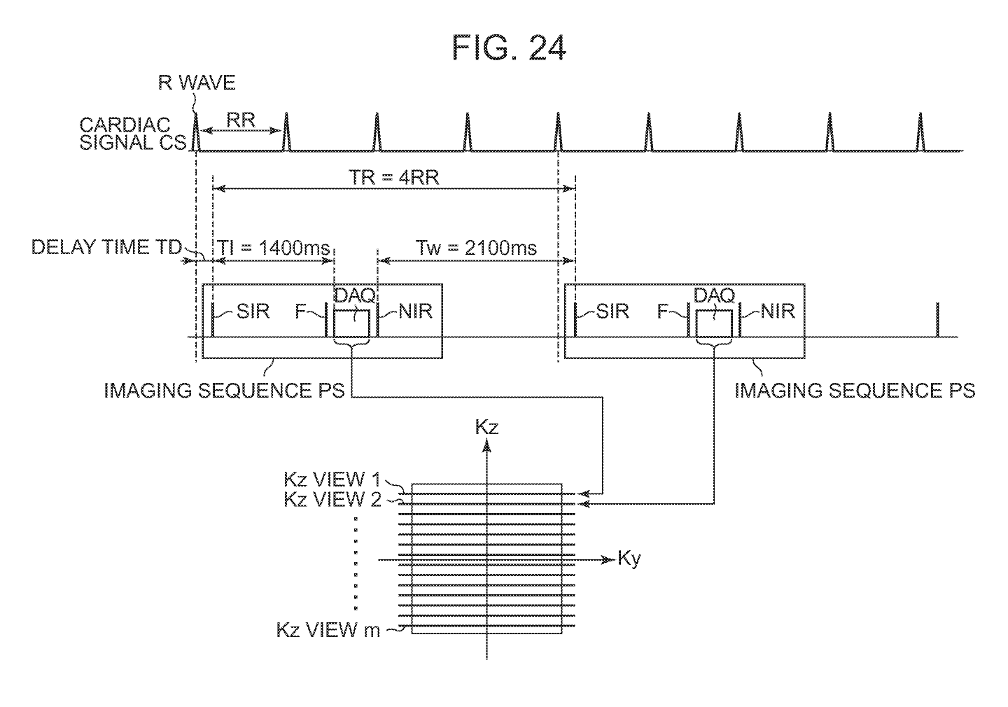

Accordingly, TI=1400 ms and Tw=2100 ms are obtained by executing the processes of Steps ST3 and ST4. FIG. 24 shows an imaging sequence PS where TI=1400 ms and Tw=2100 ms. Incidentally, since n=4, substituting n=4 into the Equation 2 yields TR=4RR.

After the waiting time Tw is determined, the flow proceeds to Step ST20 (refer to FIG. 3), where k-space data is acquired in accordance with the imaging sequence PS shown in FIG. 24, and the flowchart is ended.

A description will at last be made about where TI=1300 ms.

(3) When TI is determined as TI=1300 ms in Step ST3:

When TI is determined to be TI=1300 ms in Step ST3, the flow proceeds to Step 40a (refer to FIG. 8) as with the case where TI=1500 ms and 1400 ms.

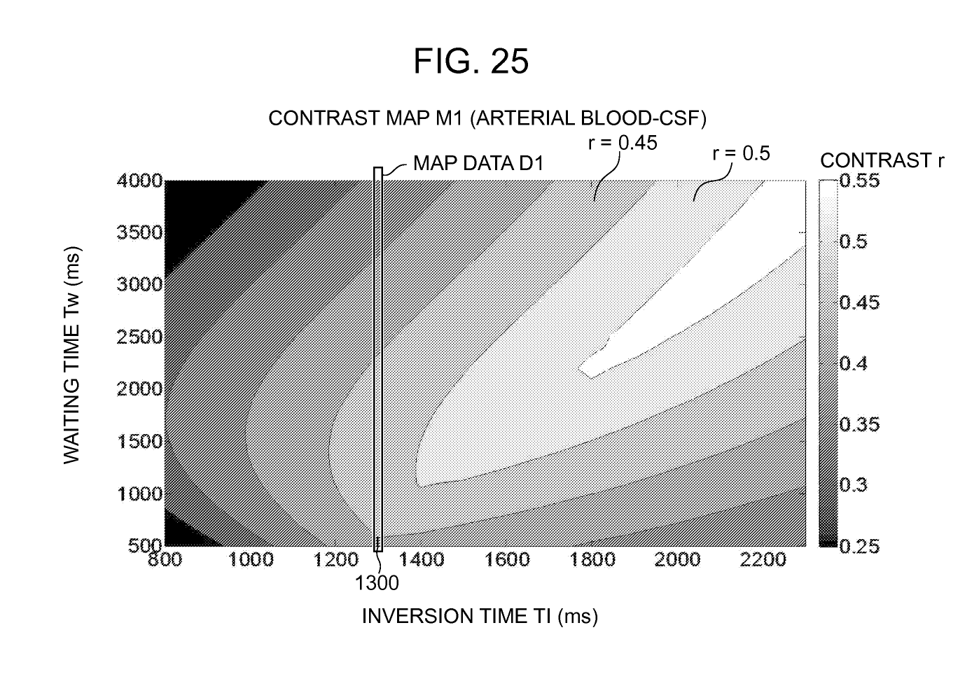

In Step 40a, the waiting time determining means 93 extracts map data at the inversion time TI=1300 (ms) determined in Step ST3 from the contrast map M1 (refer to FIG. 5). The extracted map data D1 is shown in FIG. 25. After the extraction of the map data D1, the flow proceeds to Step 40b.

In Step 40b, the waiting time determining means 93 judges whether or not the contrast r that satisfies r≧0.5 (refer to the Equation 6) is contained within the map data D1. Referring to FIG. 25, the map data D1 crosses a region of a contrast 0.45, but does not cross a region of a contrast 0.5. Thus, the contrast r that satisfies r≧0.5 is not contained in the map data D1. In this case, the flow proceeds to Step 40c.

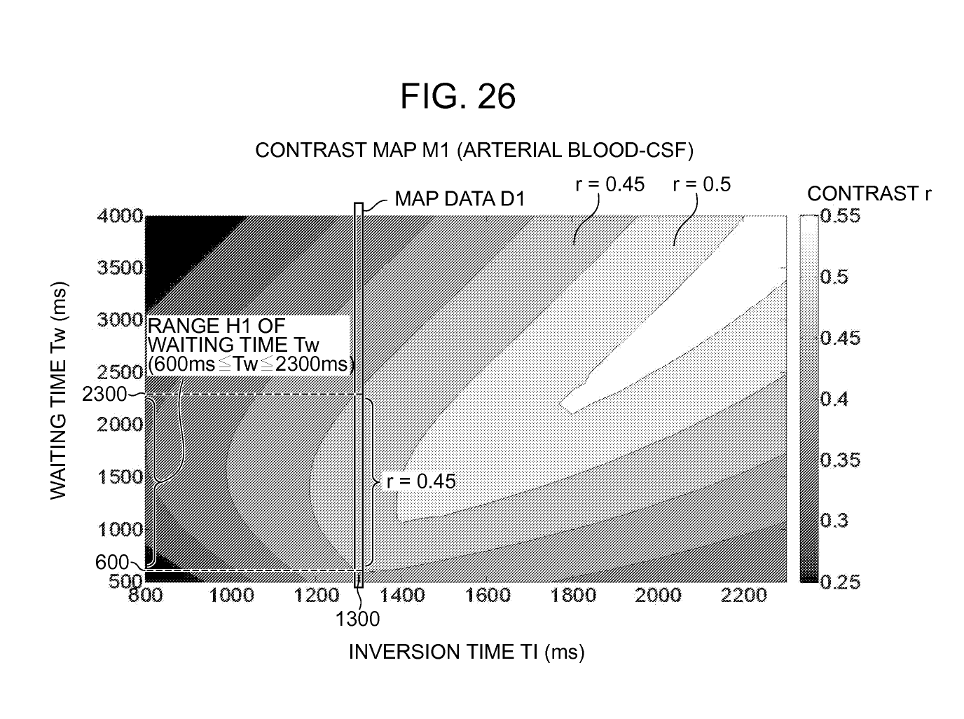

In Step 40c, the waiting time determining means 93 first determines the maximum value of the contrast r from the map data D1. Referring to FIG. 25, the maximum value of the contrast r in the map data D1 is 0.45. After the maximum value 0.45 of the contrast r is determined, the waiting time determining means 93 specifies a range H1 of a waiting time Tw corresponding to the maximum value 0.45 of the contrast r. FIG. 26 shows the range H1 of the waiting time Tw corresponding to r=0.45. The range H1 of the waiting time Tw is taken to be the following range herein:

600 (ms)≦Tw≦2300 (ms) Equation 22

After the range H1 of the waiting time Tw in the contrast map M1 is obtained, the flow proceeds to Step 40e.

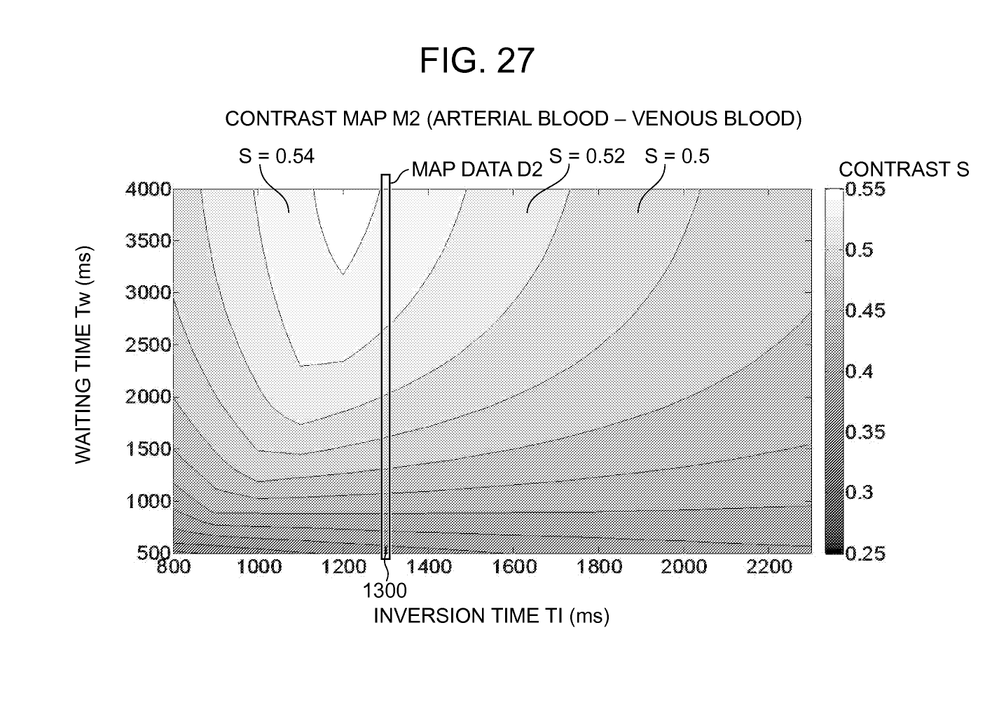

In Step 40e, the waiting time determining means 93 extracts map data at the inversion time TI=1300 (ms) determined in Step ST3 from the contrast map M2 (refer to FIG. 6). The extracted map data D2 is shown in FIG. 27. After the extraction of the map data D2, the flow proceeds to Step 40f.

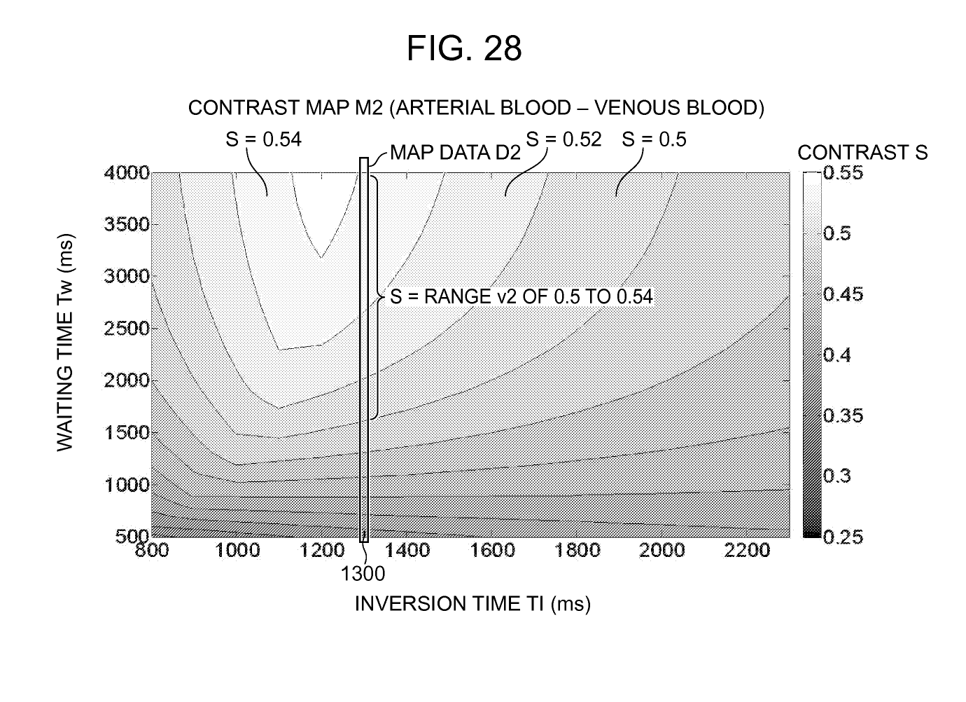

In Step 40f, the waiting time determining means 93 judges whether or not the contrast s that satisfies s≧0.5 (refer to Equation 9) is contained in the map data D2. Referring to FIG. 27, it is understood that the map data D2 crosses regions of contrasts 0.5 to 0.54. Thus, the contrast s that satisfies s≧0.5 is contained in the map data D2. FIG. 28 shows a range v2 of s=0.5 to 0.54 contained in the map data D2. Since the contrast s that satisfies s≧0.5 is contained in the map data D2, the flow proceeds to Step 40h.

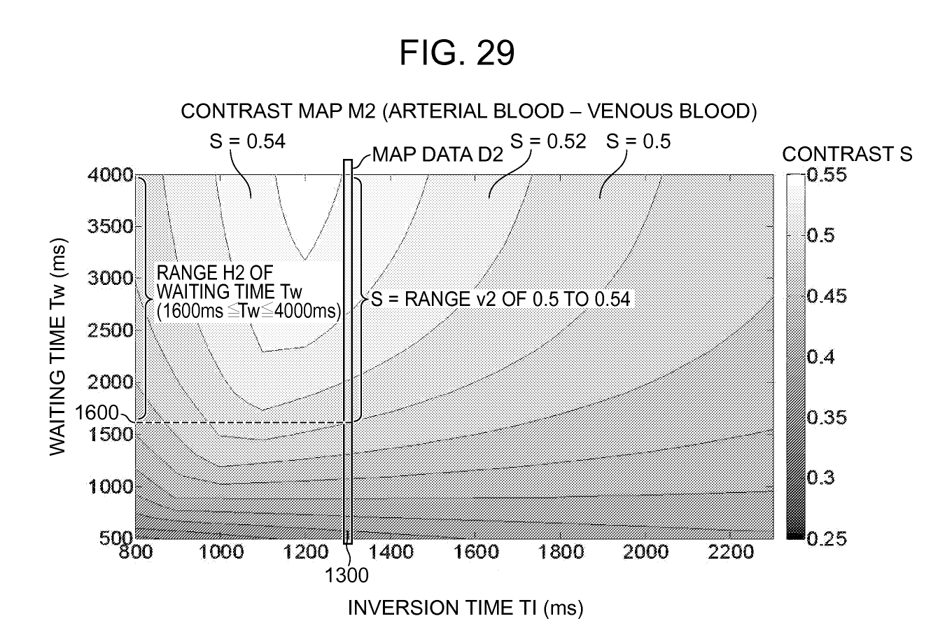

In Step 40h, the waiting time determining means 93 specifies a range H2 of a waiting time Tw corresponding to the range v2 of s=0.5 to 0.54. FIG. 29 shows the range H2 of the waiting time Tw corresponding to the v2 of s=0.5 to 0.54. The range H2 of the waiting time Tw is taken to be the following range herein:

1600 (ms)≦Tw≦4000 (ms) Equation 23

After the range H2 of the waiting time Tw in the contrast map M2 is obtained, the flow proceeds to Step 40i.

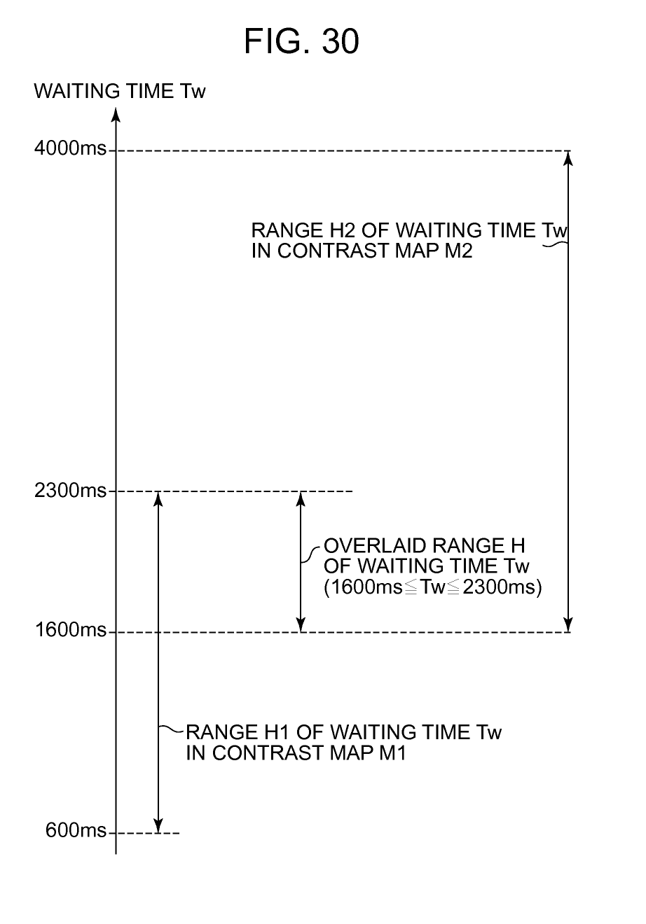

In Step 40i, the waiting time determining means 93 judges whether or not an overlaid part exists between the range H1 (refer to FIG. 26) of the waiting time Tw in the contrast map M1 and the range H2 (refer to FIG. 29) of the waiting time Tw in the contrast map M2. The range H1 of the waiting time Tw in the contrast map M1, and the range H2 of the waiting time Tw in the contrast map M2 are shown in FIG. 30 by comparison. In FIG. 30, the waiting time Tw overlaps within the range of 1600 ms≦Tw≦2300 ms. Therefore, the flow proceeds to Step 40k.

In Step 40k, the waiting time determining means 93 determines the overlapped range H of the waiting time Tw as a time range usable as a waiting time Tw. The waiting time Tw overlaps within the range of 1600 ms≦Tw≦2300 ms herein. Thus, the time range H usable as the waiting time Tw becomes the following range:

1600 ms≦Tw≦2300 ms Equation 24

When the time range usable as the waiting time Tw is determined, the flow of Step ST40 is ended. When Step ST40 is terminated, the flow proceeds to Step ST41 (refer to FIG. 7).

In Step ST41, the waiting time determining means 93 determines a waiting time Tw at the execution of an imaging sequence PS from the time range usable as the waiting time Tw. A method of determining the waiting time Tw will be described below.

When RR and Ta are taken to be RR=1000 ms and Ta=500 ms as previously described, the waiting time Tw can be represented by Equation 14. Equation 14 is shown again below.

Tw=1000n−(TI+500) Equation 14

Here, the inversion time TI has been calculated as TI=1300 ms in Step ST3. Thus, substituting TI=1300 ms into Equation 14 yields the following equation:

$$\begin{matrix}Tw = 1000 n - ( TI + 500 ) \\ = 1000 n - ( 1300 + 500 ) \\ = 1000 n - 1800\end{matrix} \qquad Equation 25$$

Further, in Step ST40, the time range H usable as the waiting time Tw has been determined as 1600 ms≦Tw≦2300 ms (refer to Equation 24). Thus, the waiting time Tw expressed in Equation 25 is required to satisfy the following equation:

1600 ms≦Tw=1000n−1800≦2300 ms Equation 26

The waiting time determining means 93 determines n that satisfies Equation 26. n that satisfies Equation 26 is n=4 and 5. Substituting n=4 into Equation 25 yields the following value as the waiting time Tw:

$$\begin{matrix}Tw = 1000 n - 1800 \\ = 1000 \times 4 - 1800 \\ = 2200 ms\end{matrix}$$

On the other hand, when n=5 is substituted into Equation 25, the waiting time Tw becomes the following value:

$$\begin{matrix}Tw = 1000 n - 1800 \\ = 1000 \times 5 - 1800 \\ = 3200 ms\end{matrix}$$

Accordingly, the two waiting times Tw=2200 ms and Tw=3200 ms are obtained. Thus, when the waiting time Tw that satisfies Equation 26 exists in plural form, the minimum waiting time Tw (=2200 ms) is determined as the waiting time Tw at the execution of the imaging sequence PS in such a manner that a scan time becomes short.

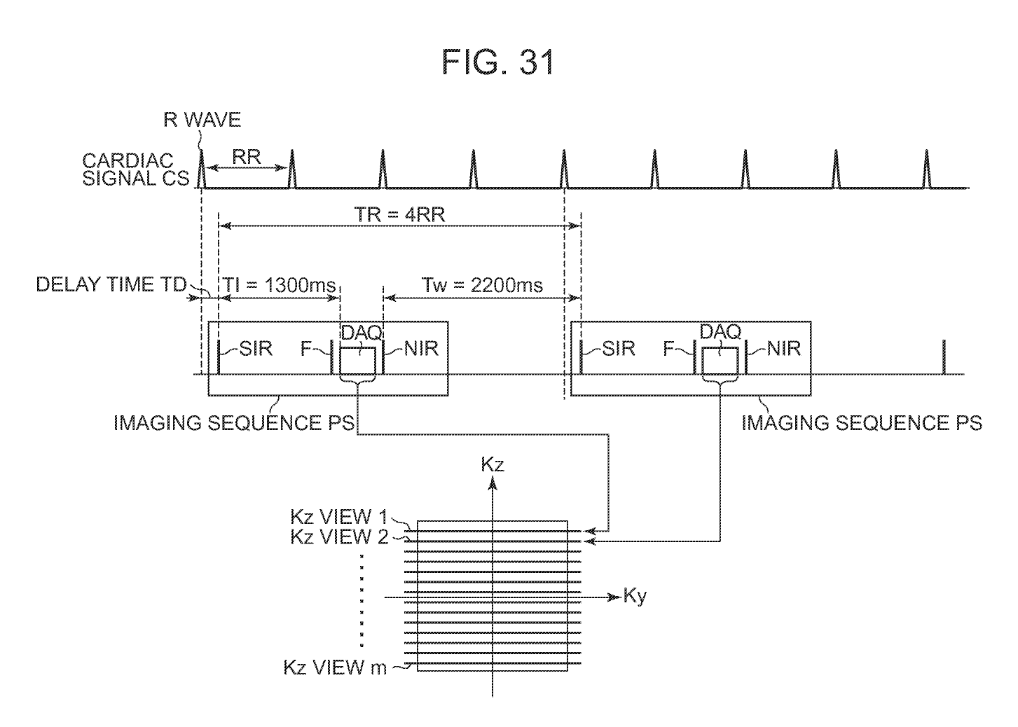

Accordingly, TI=1300 ms and Tw=2200 ms are obtained by executing the processes of Steps ST3 and ST4. FIG. 31 shows an imaging sequence PS where TI=1300 ms and Tw=2200 ms. Incidentally, since n=4, substituting n=4 into Equation 2 yields TR=4RR.

After the waiting time Tw is determined, the flow proceeds to Step ST20 (refer to FIG. 3), where k-space data is acquired in accordance with the imaging sequence PS shown in FIG. 31, and the flowchart is ended.

When TI=1300 ms, the contrast of r≧0.5 is not contained in the map data D1 extracted from the contrast map M1. Therefore, the maximum value 0.45 of the contrast r is determined from the map data D1 to specify a range H1 (600 ms≦Tw≦2300 ms) of a waiting time Tw corresponding to the maximum value 0.45 of the contrast (refer to FIG. 26). Thereafter, an overlaid range H of the waiting time Tw is obtained (refer to FIG. 30). The waiting time Tw is determined from this range H. Accordingly, the value of the contrast r can be set to the maximum value allowable when TI=1300 ms.

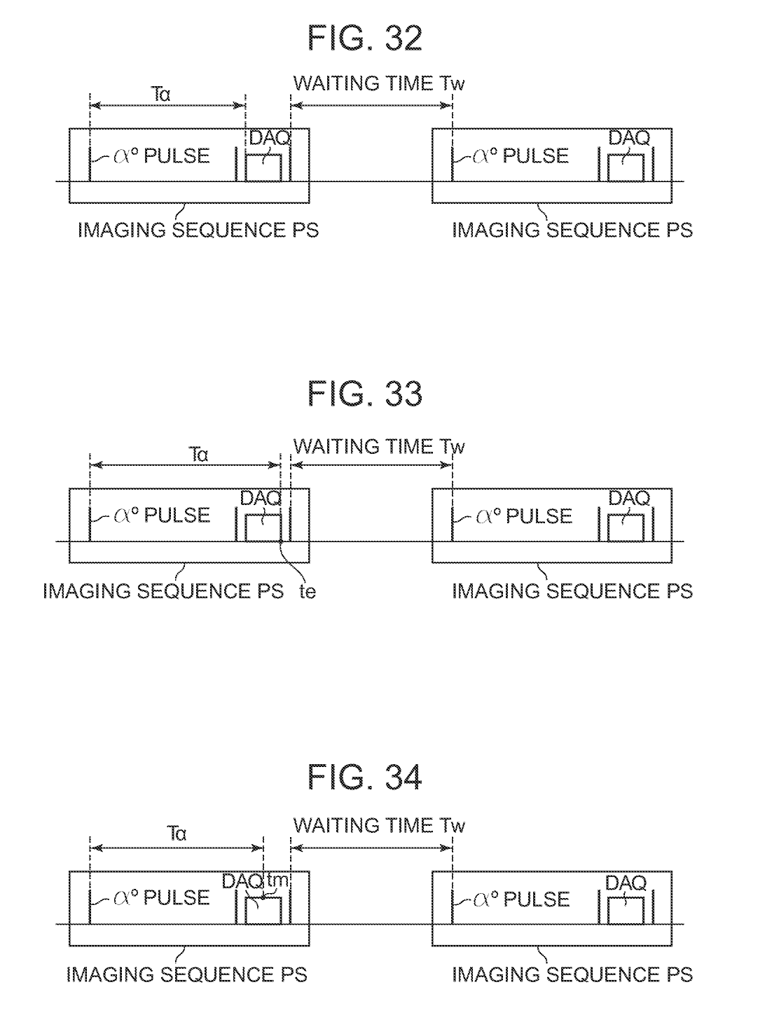

Incidentally, in the exemplary embodiment, the imaging sequence PS has the inversion pulse SIR (i.e., RF pulse of which the flip angle is 180°). The disclosure is however not limited to the inversion pulse SIR, but can use an α° pulse (where α: arbitrary angle). FIG. 32 shows an example of an imaging sequence PS where the α° pulse is used. In FIG. 32, the time between the α° pulse and the data acquisition sequence DAQ is expressed in “Tα”. In this case, the time Tα may be calculated based on the flow velocity v of the arterial blood as with the inversion time TI. Further, the waiting time Tw can be determined using the contrast map that defines the correspondence relation between the time Tα, the waiting time Tw and the contrast r (or contrast s).

Further, the time Tα may be set as the time between an α° pulse and a completion time point te of a data acquisition sequence DAQ as shown in FIG. 33. Alternatively, the time tα may be set as the time between an α° pulse and a time point tm halfway of execution of a data acquisition sequence DAQ as shown in FIG. 34.

Incidentally, in the exemplary embodiment, the waiting time Tw is determined using the two contrast maps M1 and M2, but may be determined using only either one of the contrast maps. For example, when it is important to increase the contrast between the arterial blood and the venous blood, the waiting time Tw may be determined using only the contrast map M2. On the other hand, when it is important to increase the contrast between the arterial blood and CSF, the waiting time Tw may be determined using only the contrast map M1.

Further, in the exemplary embodiment, the imaging sequence PS is provided with the selective inversion pulse SIR, the fat suppression pulse F, the data acquisition sequence DAQ, and the nonselective inversion pulse NIR. The imaging sequence PS may however have pulses other than the selective inversion pulse SIR, the fat suppression pulse F and the nonselective inversion pulse NIR, or have a sequence different from the data acquisition sequence DAQ. Further, the imaging sequence PS is provided with the fat suppression pulse F and the nonselective inversion pulse NIR, but may not include these pulses F and NIR.

Although the exemplary embodiment has described the example in which the arterial blood is imaged, the systems and methods described herein can be applied even to the case where the venous blood is imaged.

While the disclosure has been described specifically on the basis of the exemplary embodiment, the present invention is not limited to the exemplary embodiments referred to above. It is needless to say that various changes can be made thereto within the scope not departing from the gist thereof.

1. A magnetic resonance system configured to repeatedly execute imaging sequences each having a first RF pulse for flipping each spin in a region containing blood, and a data acquisition sequence acquiring data of the blood from the region, the magnetic resonance system comprising:
a storage unit configured to store a correspondence relation between a contrast between the blood and a background tissue, a first time taken from the first RF pulse to the data acquisition sequence, and a second time taken from a completion of an imaging sequence to a start of a next imaging sequence;
first determining means configured to determine the first time used when the imaging sequence is repeatedly executed, based on a flow velocity of the blood; and
second determining means configured to determine the second time used when the imaging sequence is repeatedly executed, based on the first time determined by the first determining means and the correspondence relation.

2. The magnetic resonance system according to claim 1, wherein the storage unit is configured to store a first map that defines a correspondence relation between a contrast between the blood and a first background tissue, a first time taken from the first RF pulse to the data acquisition sequence, and a second time taken from a completion of an imaging sequence to a start of a next imaging sequence, and
wherein the second determining means is configured to determine a time range usable as the second time, based on the first time determined by the first determining means and the first map, and is configured to determine the second time used when the imaging sequence is repeatedly executed, from the time range.

3. The magnetic resonance system according to claim 2, wherein the blood is arterial blood, and
wherein the first background tissue is venous blood.

4. The magnetic resonance system according to claim 2, wherein the second determining means is configured to specify first data at the first time determined by the first determining means from the first map, and is configured to determine a time range usable as the second time, based on the first data.

5. The magnetic resonance system according to claim 4, wherein the storage unit is configured to store a second map that defines a second correspondence relation between a contrast between the blood and a second background tissue, a first time taken from the first RF pulse to the data acquisition sequence, and a second time taken from a completion of an imaging sequence to a start of a next imaging sequence, and
wherein the second determining means is configured to specify second data at the first time determined by the first determining means from the second map, and is configured to determine a time range usable as the second time, based on the first data and the second data.

6. The magnetic resonance system according to claim 5, wherein the second determining means is configured to specify a first range of the second time corresponding to a contrast greater than or equal to a first predetermined value, based on the first data,
wherein the second determining means is configured to specify a second range of the second time corresponding to a contrast greater than or equal to a second predetermined value, based on the second data, and
wherein the second determining means is configured to determine a time range usable as the second time, based on the first range of the second time and the second range of the second time.

7. The magnetic resonance system according to claim 6, wherein when an overlaid range of the second time exists between the first range of the second time and the second range of the second time, the second determining means is configured to determine the overlaid range of the second time as a time range usable as the second time.

8. The magnetic resonance system according to claim 6, wherein when the overlaid range of the second time does not exist between the first range of the second time and the second range of the second time, the second determining means is configured to determine the first range as a time range usable as the second time.

9. The magnetic resonance system according to claim 5, wherein the second background tissue is cerebrospinal fluid.

10. The magnetic resonance system according to claim 2, wherein the second determining means is configured to determine the second time used when the imaging sequence is repeatedly executed, from the time range usable as the second time on the basis of a cardiac rate or an RR interval and the first time determined by the first determining means.

11. The magnetic resonance system according to claim 1, wherein the first time is a time taken from the first RF pulse to a starting point of time of the data acquisition sequence.

12. The magnetic resonance system according to claim 11, wherein the first RF pulse is an inversion pulse, and the first time is an inversion time.

13. The magnetic resonance system according to claim 1, wherein the imaging sequence has a second RF pulse between the first RF pulse and the data acquisition sequence.

14. The magnetic resonance system according to claim 13, wherein the second RF pulse is a fat suppression pulse.

15. The magnetic resonance system according to claim 1, wherein a sequence for determining the flow velocity of the blood is executed.

16. A program applied to a magnetic resonance system which is configured to repeatedly execute imaging sequences each having a first RF pulse for flipping each spin in a region containing blood, and a data acquisition sequence acquiring data of the blood from the region, and which is configured to store a correspondence relation between a contrast between the blood and a background tissue, a first time taken from the first RF pulse to the data acquisition sequence, and a second time taken from a completion of an imaging sequence to a start of a next imaging sequence, the program configured to cause a computer to execute:
a first determining process to determine the first time used when the imaging sequence is repeatedly executed, based on a flow velocity of the blood; and
a second determining process to determine the second time used when the imaging sequence is repeatedly executed, based on the first time determined by the first determining means and the correspondence relation.

17. The program according to claim 16, wherein the blood is arterial blood.

18. The program according to claim 16, wherein the imaging sequence has a second RF pulse between the first RF pulse and the data acquisition sequence.

19. The program according to claim 18, wherein the second RF pulse is a fat suppression pulse.

20. The program according to claim 16, wherein the first RF pulse is an inversion pulse, and the first time is an inversion time.
